(19) United States
(12) Patent Application Publication
(10) Pub. No.: US 2024/0296958 A1
(43) Pub. Date: Sep. 5, 2024

(54) SYSTEMS AND METHODS FOR PREDICTION, PREVENTION AND MANAGEMENT OF CHRONIC AND AUTOIMMUNE DISEASES

(71) Applicant: SWEETCH HEALTH LTD., Ramat Gan (IL)

(72) Inventors: Dana CHANAN, Tel Aviv (IL); Ilan SINAI, Pardes Hana (IL); Marina ASHEROV, Yavne (IL); Nir ATSHULER, Parles Hana (IL); Eran KROITORU, Herzliya (IL); Yael PELEG, Beer Yakov (IL); Yossi BAHAGON, Jerusalem (IL); Garold FUKS, Hod-Hasharon (IL)

(21) Appl. No.: 18/431,915

(22) Filed: Feb. 2, 2024

Related U.S. Application Data
(63) Continuation of application No. PCT/IB2022/057335, filed on Aug. 5, 2022.
(60) Provisional application No. 63/229,616, filed on Aug. 5, 2021.

(51) Int. Cl. G16H 50/30; A61B 5/16; G16H 10/60; G16H 40/67

(52) U.S. Cl. CPC G16H 50/30; A61B 5/165; G16H 10/60; G16H 40/67

(57) ABSTRACT

Systems and methods for prediction, prevention and management of chronic and autoimmune diseases. Inputs containing data are received from a user via at least one of a mobile device, an application provided thereon, or one or more connected devices. Data is processed via one or more user profiling engines and one or more user context engines. Based on the user profiling engine(s), a digital blueprint of the user is generated. Also, via an incremental change engine, incremental change programs (ICPs) are generated based at least in part on data processed from the user context engine(s), the digital blueprint of the user, and a predefined set of goals. An interaction engine is designed to assemble based at least in part on user goals, the plurality of ICPs, and the digital blueprint, a specific interaction to be delivered. Thereafter, the specific interaction is delivered to the user in accordance with an optimal time and domain of intervention.

### CROSS REFERENCE TO RELATED APPLICATION(S)

This application is a Continuation of International Patent Application No. PCT/IB2022/057335, filed Aug. 5, 2022, which claims priority to U.S. Provisional Patent Application No. 63/229,616, filed Aug. 5, 2021, the contents of which are hereby incorporated by reference in their entirety.

### BACKGROUND

### Field

The present disclosure is generally related to a mobile-health platform that utilizes Artificial Intelligence to increase patient engagement and compliance with vital disease management and to provide health promotion recommendations. Disclosed are systems and methods that process data received via at least one of a mobile device, an application provided thereon, or one or more connected devices, and deliver a specific interaction to a user in order to increase compliance.

### Description of Related Art

Chronic diseases now affect approximately half of the US population, cause 7 in 10 deaths, and accounts for roughly 86% of US health care expenditure. The clinical and financial outcomes associated with patients having one or more chronic diseases are profoundly affected beneficially by better adherence to healthy life habits and disease management.

In 2016, the Centers for Disease Control and Prevention (CDC) published a study on the relationship between Inadequate physical activity and healthcare expenditures in the United States. The researchers found that individuals who didn't exercise spent, on average, $1,313 more on healthcare every year compared to active adults. To put things in perspective, according to the CDC, 11.1% of healthcare costs in the United States are related to inadequate physical activity. In 2018, the United States spent $3.6 trillion on healthcare. Inactivity, therefore, is responsible for $333 billion in healthcare costs.

Effective therapies require frequent, individualized interventions that extend beyond the hospital and clinic to reach patients in their day-to-day lives. However, a mismatch currently exists between what the health care system and human-dependent chronic disease prevention and management programs are equipped to provide, and the interventions necessary to effectively address the chronic disease epidemic burden. Improving clinical outcomes requires overcoming the “last mile problem” involving patient behavior, as even the most appropriate medical treatment will be effective only if the patient follows through on it.

### SUMMARY

This disclosure provides a clinically-validated mobile-health platform that utilizes Artificial Intelligence to significantly increase patient engagement and compliance with vital disease management and health promotion recommendations.

It is an aspect of this disclosure to provide systems and methods for prediction, prevention and management of chronic and autoimmune diseases. As described herein, a plurality of inputs containing data are received from a user via at least one of a mobile device, an application provided thereon, or one or more connected devices. Data is processed via one or more user profiling engines and one or more user context engines. Based on the one or more user profiling engines, a digital blueprint of the user is generated. Also, via an incremental change engine, a plurality of incremental change programs (ICPs), based at least in part on data processed from the one or more user context engines, the digital blueprint of the user, and a predefined set of goals, are generated. An interaction engine is designed to assemble, based at least in part on the plurality of ICPs, the predefined set of one or more user goals, and the digital blueprint of the user, a specific interaction to be delivered to the user. Thereafter, the specific interaction to the user, in accordance with an optimal time and domain of intervention, is delivered.

Another aspect provides a system for prediction, prevention and management of chronic and autoimmune diseases. The system includes: memory storing computer program instructions; and one or more processors configured to execute the computer program instructions to: receive a plurality of inputs containing data from a user via at least one of a mobile device, an application provided thereon, or one or more connected devices; process the data via one or more user profiling engines and one or more user context engines; generate, based on the one or more user profiling engines, a digital blueprint of the user; generate, by an incremental change engine, a plurality of incremental change programs (ICPs), based at least in part on data processed from the one or more user context engines, the digital blueprint of the user, and a predefined set of one or more user goals; assemble, by an interaction engine, based at least in part on the plurality of ICPs, the predefined set of one or more user goals, the digital blueprint of the user, a specific interaction to be delivered to the user; and deliver the specific interaction to the user in accordance with an optimal time and domain of intervention.

Yet another aspect provides a non-transitory computer readable medium having instructions recorded thereon for prediction, prevention and management of chronic and autoimmune diseases. The instructions when executed by a computer having at least one programmable processor cause operations including: receiving a plurality of inputs containing data from a user via at least one of a mobile device, an application provided thereon, or one or more connected devices; processing the data via one or more user profiling engines and one or more user context engines; generating, based on the one or more user profiling engines, a digital blueprint of the user; generating, by an incremental change engine, a plurality of incremental change programs (ICPs), based at least in part on data processed from the one or more user context engines, the digital blueprint of the user, and a predefined set of one or more user goals; assembling, by an interaction engine, based at least in part on the plurality of ICPs, the predefined set of one or more user goals, the digital blueprint of the user, a specific interaction to be delivered to the user; and delivering the specific interaction to the user in accordance with an optimal time and domain of intervention.

Other aspects, features, and advantages of the present disclosure will become apparent from the following detailed description, the accompanying drawings, and the appended claims.

### BRIEF DESCRIPTION OF THE DRAWINGS

FIG. 1 shows a high level diagram illustrating an example configuration of a system for performing one or more aspects described herein, according to at least one embodiment.

FIG. 2 illustrates examples of pre-processing steps that may be performed by the system and as part of the method, in accordance with an embodiment.

FIG. 3 illustrates examples of processing steps that may be performed by the system and as part of the method, in accordance with an embodiment.

FIG. 4 illustrates examples of post-processing steps that may be performed by the system and as part of the method, in accordance with an embodiment.

FIGS. 5 and 6 illustrate examples of code structure diagrams for models and ensembles, in accordance with embodiments herein.

FIG. 7 illustrates an example of a Sweetch Intervention Package in accordance with an embodiment.

FIG. 8 illustrates an example of Sweetch personalized messaging in accordance with an embodiment.

FIG. 9 illustrates an example of a display showing an exemplary output to a user in accordance with an embodiment.

FIG. 10 illustrates examples of display screens showing features related to Activity Management for a user in accordance with an embodiment.

FIG. 11 illustrates examples of display screens showing features related to Weight management in accordance with an embodiment.

FIG. 12 illustrates examples of display screens showing features related to Diet and Nutrition in accordance with an embodiment.

FIG. 13 illustrates examples of display screens showing features related to Medication Management in accordance with an embodiment.

FIG. 14 illustrates examples of display screens showing features related to Sweetch PRO Module in accordance with an embodiment.

FIG. 15 illustrates an example of a connected device module of a Sweetch Intervention Package in accordance with an embodiment.

FIG. 16 illustrates an example of a Sweetch management dashboard in accordance with an embodiment.

FIG. 17 illustrates an example of Educational materials in display screen samples in accordance with an embodiment.

FIG. 18 illustrates an example of Sweetch gamification features on display screens in accordance with an embodiment.

FIG. 19 illustrates an example of In-app Patient Data on display screens in accordance with an embodiment.

FIG. 20 illustrates an example of a Sweetch Offering for COVID-19 and/or other pandemic occurrences in accordance with an embodiment.

FIG. 21 illustrates an example of a Block Diagram of modules involved in innovation points discussed in accordance with an embodiment.

FIG. 22 illustrates an example of Sweetch prediction flow in accordance with an embodiment.

FIG. 23 illustrates an example of Sweetch intervention flow in accordance with an embodiment.

FIG. 24 illustrates an example of a block diagram of Sweetch logic tier architecture in accordance with an embodiment.

FIG. 25 illustrates an example of Daily Messages RL AI in accordance with an embodiment.

FIG. 26 illustrates an example of a Client API Server in accordance with an embodiment.

FIG. 27 illustrates an example of a Location Server in accordance with an embodiment.

FIG. 28 illustrates an example of a BI Server in accordance with an embodiment.

FIG. 29 illustrates an example of a Sweetch AWS architecture in accordance with an embodiment.

FIG. 30 shows exemplary possible combinations of prediction results with regards to training an HMO dataset, in accordance with an embodiment.

FIG. 31 illustrates examples of sentiment(s) and daily messages provided by a reinforcement learning AI algorithm in accordance with an embodiment.

FIG. 32 illustrates an exemplary embodiment of the system, in accordance with this disclosure.

FIG. 33 illustrates a clustering system in accordance with an embodiment.

FIG. 34 illustrates another example of pre-processing steps that may be performed by the system and as part of the method, in accordance with another embodiment.

FIG. 35 illustrates an example of habits and lifeprints that may be detected by the system in accordance with an embodiment.

FIG. 36 illustrates an example of an interactions engines architecture of the system shown in FIG. 32, in accordance with an embodiment.

FIG. 37 shows an implementation of a generic logic of the system in accordance with an embodiment.

FIG. 38 illustrates an example flow of SAIL (Sweetch Artificial Intelligence Language) implementation in accordance with an embodiment.

### DETAILED DESCRIPTION OF THE PREFERRED EMBODIMENT(S)

### Overview

As evident by the drawings and below description, this disclosure relates to an intervention platform that is an artificial intelligence solution that monitors and analyzes the individual's personal, environmental and behavioral digital markers. The intervention platform learns the user's various life habits by applying advanced analytics to data points extracted from the user's smartphone sensors and/or connected devices in order to send personal, contextual, real-time, recommendations promoting an increase in physical activity, weight loss, and enhancement of health and nutrition. Additionally, the system provides statistics users behavior and insights into how these behaviors influence his health. The platform considers the current state of the patient and uses a wide range of algorithms to select the required intervention to achieve maximum efficiency. System recommendations and actions are based on best practices in the fields of coaching and behavioral change science implemented in a fully automatic way. In addition, the disclosed system(s) and method(s) continuously optimize(s) the user's goals and messages, so that such goals and messages are in the context, time, place, and tone-of-voice that increase probability of patient response. There are multiple parameters that influence the intervention program. For example, patients age, gender, behavioral change state (contemplation, preparation, action and maintenance), activity program level, actual activity, locations, availability, daily habits, day of week, weather, dietary intake, physiological parameters (e.g. glucose levels, when available), etc. These capabilities are essential for continuously refining the interaction with patients to improve intervention effectiveness and adherence. Sweetch platform includes several users and machine interfaces, responsible for all the communication with the users and also an analytic interaction engine, responsible for defining the user activity and response rules, user advances, etc.

### Data Input & Analysis

As described in embodiments, the disclosed systems and methods (also referred to herethroughout as “Sweetch”) captures and analyses data from three sources:
Smartphone-based digital data streams
Digital data streams from other connected devices
Patient-generated/reported data

Smartphone originated data is used to create real-time insights about the user's current context, as well as broader insights on the patient's life habits, in accordance with embodiments herein. These include, but are not limited to—using GPS data for determining geolocation, using the accelerometer data for monitoring activity, using the combination of GPS and accelerometer to determine motion context (e.g., driving), using WIFI connection data for further validating location context (e.g., home vs. work, trusted locations), using GPS data for gathering weather information, understating availability patterns via calendar availability.

Raw data is analyzed to understand the patient's real-time context and availability (patient moments), in accordance with embodiments herein. For example, the patient is at home, the patient is at work, the patient entered a coffee shop, beginning of a walking session was detected, the patient is idle at home, etc. Each moment detected by Sweetch allows timely response and the ability to deliver a truly personalized experience.

Based on the moments collected, the system is configured to create a baseline map of the patient's life habits, i.e., how does a typical Sunday looks like, how does a typical weekend day looks like, usual waking time, usual minutes walked, continuous walking per walking session, typical go-to-sleep time slot, typical time the patient spends in driving, diet patterns, etc., in accordance with embodiments herein

As will become evident by the description, data collected from connected devices includes, but it not limited to, Bluetooth enabled scale, blood glucose monitor, smart watch, Continuous Glucose Monitor (CGM) and blood pressure monitor. By collecting these data streams from device(s), the system may be configured to correlate the patient's behavior with his/her clinical outcomes, in accordance with embodiments herein. Sweetch connected scale provides insights not only about the patient's current weight and weight trends but also insights on other weight-related measures such as lean body mass and fat body mass. Together with the patient's input on his/her diet habits and activity patterns, the system creates deeper insights on the best way to keep the patient motivated and compliant in the long run.

All the data mentioned above and herethroughout may be collected and analyzed automatically and seamlessly with no reliance on the patient's active input.

In accordance with embodiments herein, patient generated data includes questionnaires/forms the patient is asked to fill/report on. For example, such questions may be based on his/her diet habits, pain, and fatigue levels. Questionnaires are adapted based on the specific health condition the solution is targeting. In accordance with embodiments, Sweetch diet questionnaire may be designed to target dietary awareness rather than “obsessive” calorie counting. Sweetch prompts for patient's active input may follow the same just-in-time-adaptive characteristics as other prompts, to increase the likelihood of the patient responding to the form presented to him/her.

Sweetch combines patient reported outcomes with objective data to further strengthen the patient's adherence. For example, informing patients and care providers on how the patient's behavior patterns are linked to clinical outcomes.

Throughout this disclosure and the drawings, the following may be referenced:

| Abbreviation | Definition |
|---|---|
| Horizon | Number of years to be forecasted |
| Conversion Record | First time conversion record |
| Conversion Year | The Calendar year of the Conversion record |
| Cut Off Year | Last Calendar year of the Training/Validation |
| T2D, T1D | Type 2/1 Diabetes |
| HR, MR, LR | High Risk group, Mid Risk, Low Risk |
| MidRiskFeaturesSet1 | MidRiskFeaturesSet1 - association of clinical risk level |
| | together with |
| | various sets of features involving considerations of data |
| | availably etc. |
| TM | Totally Missing group - patients without any A1C and FBG |
| AllRisk | Group of HR + MR + LR combined, i.e., any patient which has |
| | A1C |
| AUC | Area Under Curve - performance measure for binary |
| | classifiers which is Threshold free. |
| XGB | Algorithm model that uses boosting & trees. See also: |
| | https://en.wikipedia.org/wiki/XGBoost |
| RF | Random Forest algorithmic Model |
| Logistic | Logistic Regression Algorithmic Model |
| Boosting | A family of machine learning algorithms that convert weak |
| | learners to strong ones, iteratively learning weak classifiers |
| | with respect to a distribution and adding them to a final |
| | strong classifier. When they are added, they are typically |
| | weighted in some way that is usually related to the weak |
| | learners' accuracy. After a weak learner is added, the data |
| | weights are readjusted, known as “re-weighting”. |
| | Misclassified input data gain a higher weight and examples |
| | that are classified correctly lose weight. Thus, future weak |
| | learners focus more on the examples that previous weak |
| | learners misclassified. |
| WS features | Minimal set of features - subset of all lab measurements. It is |
| | considered de facto minimum set of features needed in order |
| | to give a prediction. |
| GT | Ground True (or ground truth) |
| SparseFeatures | Adding features sparsity to HMO data in order to fit the |
| | feature statistics of the testing\Roche data. |
| Horizon | Prediction for a specific year ahead. For example, Horizon = |
| | 2 (H2) - if the patient is converted, it will only be in the |
| | second year of the prediction. |
| Cutoff Year | The year that separates Train/Test Data from GT data; |
| | The time point where the prediction has to happen is called a |
| | cut-off point. This point is referred to in a yearly resolution. |
| ICP | Incremental Change Program |
| IE | Interaction Engine |
| BGM | Blood Glucose Monitoring |
| CGM | Continuous Glucose Monitoring |
| KDE | Kernel Density Estimation |

### System Architecture:

In the detailed description herein, numerous specific details are set forth in order to provide a thorough understanding of the disclosure. However, it will be understood by those skilled in the art that the present disclosure may be practiced without these specific details. In other instances, well-known methods, procedures, and components, modules, units and/or circuits have not been described in detail so as not to obscure the disclosure. Some features and/or elements described with respect to one embodiment may be combined with features and/or elements described with respect to other embodiments. For the sake of clarity, discussion of same or similar features and/or elements may not be repeated.

Although embodiments of the disclosure are not limited in this regard, discussions utilizing terms such as, for example, “processing,” “computing,” “calculating,” “determining,” “establishing”, “analyzing”, “checking”, or the like, may refer to operation(s) and/or process(es) of a computer, a computing platform, a computing system, or other electronic computing device, that manipulates and/or transforms data represented as physical (e.g., electronic) quantities within the computer's registers and/or memories into other data similarly represented as physical quantities within the computer's registers and/or memories or other information non-transitory storage medium that may store instructions to perform operations and/or processes. Non-transitory computer-readable media, as used herein, comprises all computer-readable media except for transitory, propagating signals. Although embodiments of the disclosure are not limited in this regard, the terms “plurality” and “a plurality” as used herein may include, for example, “multiple” or “two or more”. The terms “plurality” or “a plurality” may be used throughout the specification to describe two or more components, devices, elements, units, parameters, or the like. Unless explicitly stated, the method embodiments described herein are not constrained to a particular order or sequence. Additionally, some of the described method embodiments or elements thereof may occur or may be performed simultaneously, at the same point in time, or concurrently.

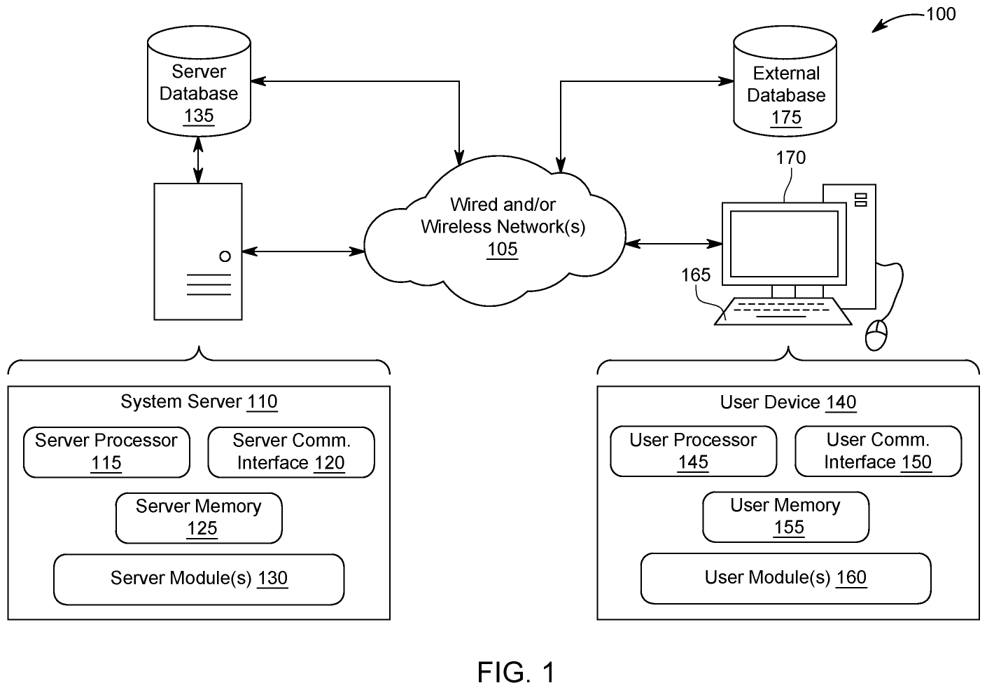

FIG. 1 shows a high level diagram illustrating an example configuration of a system 100 for performing one or more aspects of the disclosure described herein, according to at least one embodiment of the disclosure. System 100 includes network 105, which may include the Internet, one or more telephony networks, one or more network segments including local area networks (LAN) and wide area networks (WAN), one or more wireless networks, or a combination thereof. System 100 also includes a system server 110 constructed in accordance with one or more embodiments of the disclosure. In embodiments, system server 110 may be a stand-alone computer system. In other embodiments, system server 110 may include a network of operatively connected computing devices, which communicate over network 105. Therefore, system server 110 may include multiple other processing machines such as computers, and more specifically, stationary devices, mobile devices, terminals, and/or computer servers (collectively, “computing devices”). Communication with these computing devices may be, for example, direct or indirect through further machines that are accessible to the network 105.

System server 110 may be any suitable computing device and/or data processing apparatus capable of communicating with computing devices, other remote devices or computing networks, receiving, transmitting and storing electronic information and processing requests as further described herein. System server 110 is therefore intended to represent various forms of digital computers, such as laptops, desktops, workstations, personal digital assistants, servers, blade servers, mainframes, and other appropriate computers and/or networked or cloud based computing systems capable of employing the systems and methods described herein.

System server 110 may include a server processor 115 which is operatively connected to various hardware and software components that serve to enable operation of the system 100. Server processor 115 serves to execute instructions to perform various operations relating to advanced search, and other functions of embodiments of the disclosure as described in greater detail herein. Server processor 115 may be one or a number of processors, a central processing unit (CPU), a graphics processing unit (GPU), a multi-processor core, or any other type of processor, depending on the particular implementation.

System server 110 may be configured to communicate via communication interface 120 with various other devices connected to network 105. For example, communication interface 120 may include but is not limited to, a modem, a Network Interface Card (NIC), an integrated network interface, a radio frequency transmitter/receiver (e.g., Bluetooth wireless connection, cellular, Near-Field Communication (NFC) protocol, a satellite communication transmitter/receiver, an infrared port, a USB connection, and/or any other such interfaces for connecting the system server 110 to other computing devices and/or communication networks such as private networks and the Internet.

In certain implementations, a server memory 125 is accessible by server processor 115, thereby enabling server processor 115 to receive and execute instructions such a code, stored in the memory and/or storage in the form of one or more software modules 130, each module representing one or more code sets. The software modules 130 may include one or more software programs or applications (collectively referred to as the “server application”) having computer program code or a set of instructions executed partially or entirely in server processor 115 for carrying out operations for aspects of the systems and methods disclosed herein, and may be written in any combination of one or more programming languages. Server processor 115 may be configured to carry out embodiments of the present disclosure by, for example, executing code or software, and may execute the functionality of the modules as described herein.

In FIG. 1, the exemplary software modules may include a communication module, and other modules as described here. The communication module may be executed by server processor 115 to facilitate communication between system server 110 and the various software and hardware components of system 100, such as, for example, server database 135, client device 140, and/or external database 175 as described herein.

Of course, in embodiments, server modules 130 may include more or less actual modules which may be executed to enable these and other functionalities of the disclosure. The modules described herein are therefore intended to be representative of the various functionalities of system server 110 in accordance with embodiments of the disclosure. It should be noted that in accordance with various embodiments of the disclosure, server modules 130 may be executed entirely on system server 110 as a stand-alone software package, partly on system server 110 and partly on user device 140, or entirely on user device 140.

Server memory 125 may be, for example, a random access memory (RAM) or any other suitable volatile or non-volatile computer readable storage medium. Server memory 125 may also include storage which may take various forms, depending on the particular implementation. For example, the storage may contain one or more components or devices such as a hard drive, a flash memory, a rewritable optical disk, a rewritable magnetic tape, or some combination of the above. In addition, the memory and/or storage may be fixed or removable. In addition, memory and/or storage may be local to the system server 110 or located remotely.

In accordance with further embodiments of the disclosure, system server 110 may be connected to one or more database(s) 135, for example, directly or remotely via network 105. Database 135 may include any of the memory configurations as described herein, and may be in direct or indirect communication with system server 110. In embodiments, database 135 may store information relating to user documents. In embodiments, database 135 may store information related to one or more aspects of the disclosure.

As described herein, among the computing devices on or connected to the network 105 may be one or more user devices 140. User device 10 may be any standard computing device. As understood herein, in accordance with one or more embodiments, a computing device may be a stationary computing device, such as a desktop computer, kiosk and/or other machine, each of which generally has one or more processors, such as user processor 145, configured to execute code to implement a variety of functions, a computer-readable memory, such as user memory 155, a user communication interface 150, for connecting to the network 105, one or more user modules, such as user module 160, one or more input devices, such as input devices 165, and one or more output devices, such as output devices 170. Typical input devices, such as, for example, input devices 165, may include a keyboard, pointing device (e.g., mouse or digitized stylus), a web-camera, and/or a touch-sensitive display, etc. Typical output devices, such as, for example output device 170 may include one or more of a monitor, display, speaker, printer, etc.

In embodiments, user module 160 may be executed by user processor 145 to provide the various functionalities of user device 140. In particular, in embodiments, user module 160 may provide a user interface with which a user of user device 140 may interact, to, among other things, communicate with system server 110

Additionally or alternatively, a computing device may be a mobile electronic device (“MED”), which is generally understood in the art as having hardware components as in the stationary device described above, and being capable of embodying the systems and/or methods described herein, but which may further include componentry such as wireless communications circuitry, gyroscopes, inertia detection circuits, geolocation circuitry, touch sensitivity, among other sensors. Non-limiting examples of typical MEDs are smartphones, personal digital assistants, tablet computers, and the like, which may communicate over cellular and/or Wi-Fi networks or using a Bluetooth or other communication protocol. Typical input devices associated with conventional MEDs include, keyboards, microphones, accelerometers, touch screens, light meters, digital cameras, and the input jacks that enable attachment of further devices, etc.

In embodiments, user device 140 may be a “dummy” terminal, by which processing and computing may be performed on system server 110, and information may then be provided to user device 140 via server communication interface 120 for display and/or basic data manipulation. In embodiments, modules depicted as existing on and/or executing on one device may additionally or alternatively exist on and/or execute on another device. For example, in embodiments, one or more modules of server module 130, which is depicted in FIG. 1 as existing and executing on system server 110, may additionally or alternatively exist and/or execute on user device 140. Likewise, in embodiments, one or more modules of user module 160, which is depicted in FIG. 1 as existing and executing on user device 140, may additionally or alternatively exist and/or execute on system server 110.

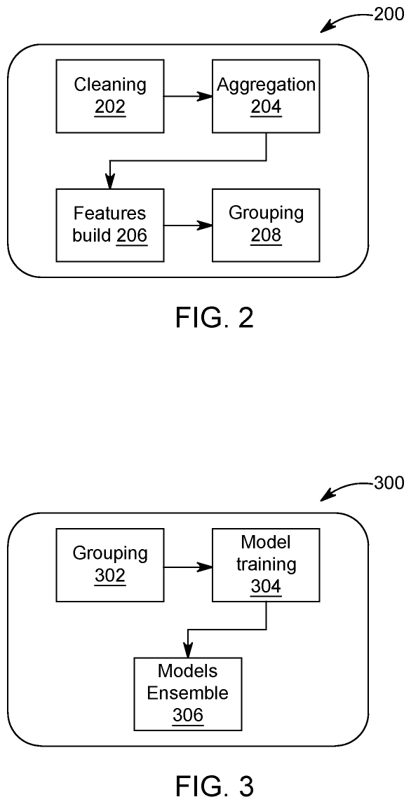

Although the later description with regards to the disclosed systems, subsystems, and functions (e.g., in FIGS. 2-4, 24, 26-28, 29, 32, 36-38) may not directly or explicitly reference the elements and features as described with reference to FIG. 1, it should be noted that such features may be tied to and/or implemented in a similar manner, using similar devices, hardware, software, etc., as described with regards to FIG. 1.

As will become evident throughout this description, the disclosure provides numerous features for the described prediction systems and methods, including, but not limited to, any of the following:

In accordance with an embodiment, Input for the system and/or method may be any of the following, part of them and/or all of them:
Blood test parameters
Demographics data, such as: age, gender, occupation, socio-demographic status, address, race
Hereditary data-Tendency for condition in the family, known gene for mutation such as, for example, BRACA
Urine test parameters
Blood Pressure parameters such as: Systolic BP, potassium, etc.
Physical Activity data
Weight Data
Dietary intake data
Medication's intake data
Any EHR related data such as former conditions, physician labeling
Any behavior data such as: physical locations, favorite visiting places and patterns, Commuting times and patterns, etc.
Sleep Data
Mood data such as “emotional feeling”, feeling depressed (y/n), etc.
Stool Data description
Cholesterol data such as: LDL, etc.
Hormone data such as: T3/T4, etc.
Liver/kidneys/Bones/Joints data such as CREATININE, GPT, etc.

In accordance with an embodiment, Input Data may include one or more of the following:
Sparse in values and/or years measurements taken
Multi years or a single year
categorial, ordinal, integers, floats-being a scalars or vectors or matrixes, etc.
Noisy
Uneven Data: varied number records per patient, uneven sampling in time, Heterogenic features sets, etc.
Correlated or not
From heterogenous or homogenous population
Single population/country or not
Unbalanced classes-Conversion is a Minority class

In accordance with an embodiment, GT data may be one or more of the following:
A1C
Glucose: Fasting, non-fasting
Conditions indications and/or conversion to other diseases (e.g., heart attack), including, but not limited to those noted later below in the list of example relevant conditions
Churn data as the GT/Label of a prediction, wherein churn may be, for example, a user stopping use of the app, stopping reporting his weight/other data, and/or termination of any other activity previously performed by the user in interaction with the system
Oral Glucose with sugar loading
Medications (e.g. transition to insulin)
W
Mix of some or all the above

In accordance with an embodiment, Hyper Parameters may be one or more of the following:
Requested Prediction Horizon in years (example: 3 years ahead)
Requested Amount/percentage of patients with risk (example: 1000 patients)

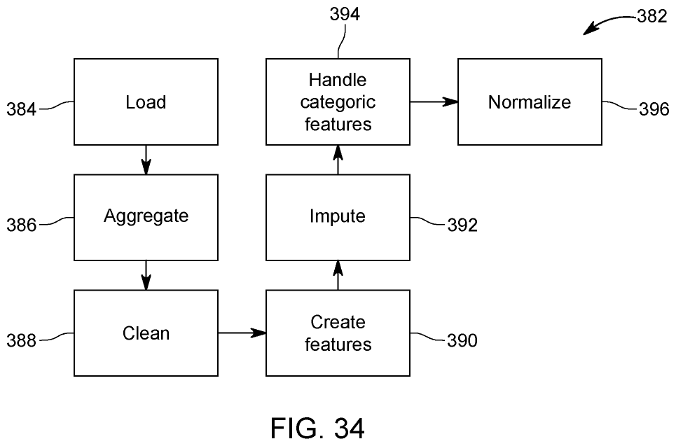

As shown in FIG. 2, the system may include one or more processors or modules designed to perform the following pre-processing steps 200 in accordance with an embodiment: Step 202: Clean—Locate and clean outliers by either removing them and/or converting to missing values; Step 204: Aggregate/Aggregation—patients' records based on grid or several grids using different metrics and normalizations; Step 206: Features—Create/Build new features based on additional information for each and every user as well as inter-patient dependencies. The features types may be, for example, categorial, ordinal, integers, floats—being a scalars or vectors or matrixes, in accordance with an embodiment. Step 208: Grouping, i.e., provide data into different groups. In an embodiment, one or both of the following optional steps may also be part of the pre-processing steps 200: Features—select features based on availability as well as feature importance, which belong to one or more of the following methods: Wrapper, Filter, and/or Embedding methods; and/or Set Ground True criteria-based on commercial request and data availability and determine conversions, if they exist. In another embodiment, the pre-processing steps may include one or more additional functions and steps, such as those shown and described with reference to FIG. 34, for example.

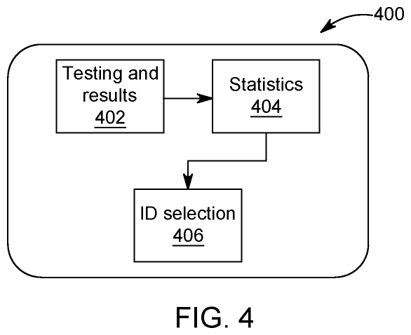

As shown in FIG. 3, the system may perform one or more of the following processing steps 300 in accordance with an embodiment: Step 302: Grouping-Split the data to different groups—this may be based on internal and/or external criteria possibly using clustering algorithm, for each group a separate model/ensemble may be implemented. An optional step in accordance with an embodiment may include Split to Train/Test/Validation groups—e.g., fully separated or using cross validation methods. Step 304: Model Training; in accordance with an embodiment, e.g., Model(s) Training may be on the Train set. An additional optional step may include Ensembles Training on the Train set. Step 306: Combining and Optimizing Models/Ensembles, creating meta-ensemble, performing cross-validation, optimizing hyper parameters, etc.

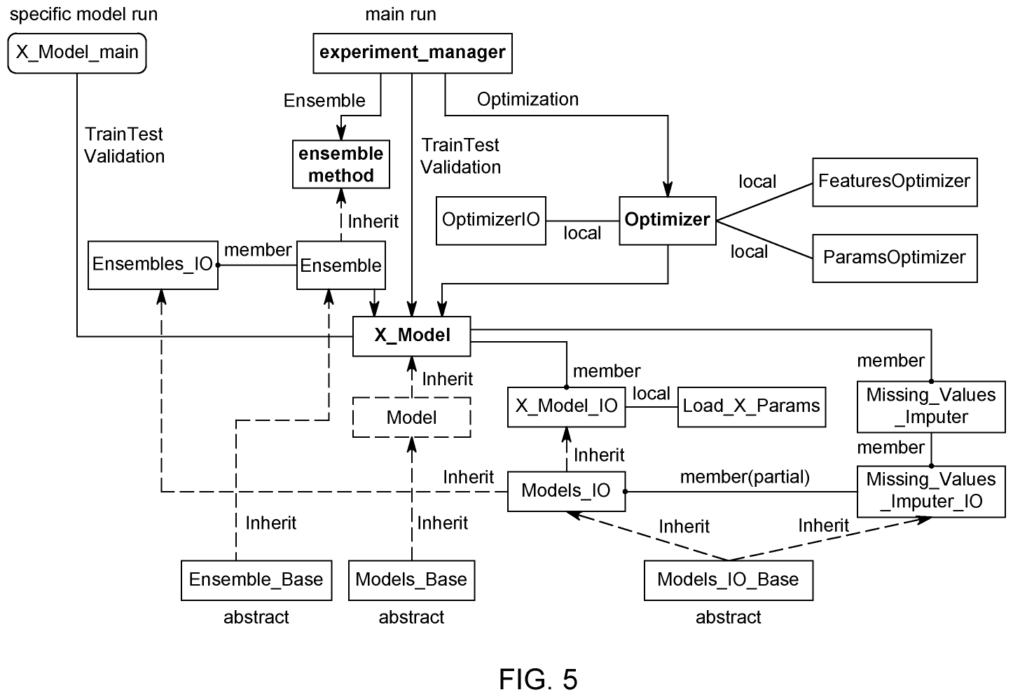

As shown in FIG. 4, the system may perform the following post-processing steps 400 with the following outputs, in accordance with an embodiment: Step 402: Testing and results, i.e., perform Testing using the Trained models and/or ensembles (as provided via completion of the processing steps 300); Step 404: Calculate Statistics on the Sets: e.g., AUC, Sensitivity, Specificity, Precision, etc., in accordance with an embodiment. Thereafter, in accordance with an embodiment, ID selection is made at step 406. In an embodiment, one or more of the following optional steps may be implemented: Rank patients, output risk and confidence per patient; Output inferences such as Comorbidities risk per patient; Output models/ensembles supporting descriptions such as features importance and other inner dependencies; and/or validation on a requested new patients' data-output is risk and confidence level.

In accordance with one embodiment, metrics and normalizations may include one or more of the following:
Min-Max normalization
Logistic normalization
Zero-one
Ramp-linear or non linear
Spline
L1, L2, Lasso

In accordance with an embodiment, Models/Ensembles may include one or more of the following:
Logistic Regression
Decision Trees: CARTS, C4.5, etc.
Linear 1D Thresholding (stump)
Hinge Penalty Model
Random Forest
XGBoost
Bagging
CSB
AdaBoost
CenterBoosting
Catboost
SVM
Metacost
K-nearest neighbors
Deep Neural network

In accordance with an embodiment, Optimizations may include one or more of the following:
Bagging
Boosting
Gradient based
Cost Based
Minority class biased w/o cost
Genetic based
Deliberate random data point annihilation based

Based on predefined scalar cost/statistical function such as AUC, F1, preferred sensitivity/specificity/Precision and/or any defined combination with prior bias setting

In accordance with an embodiment, Imputing (Missing values) may be treated by:
Substitute by zero, mean, median, max, interpolation;
Looking for different feature with closed distance; and/or
Resample on new grid/subgrid.

In accordance with an embodiment, Training may be done on one population while prediction may be done on a different population, i.e., the system may predict without re-training.

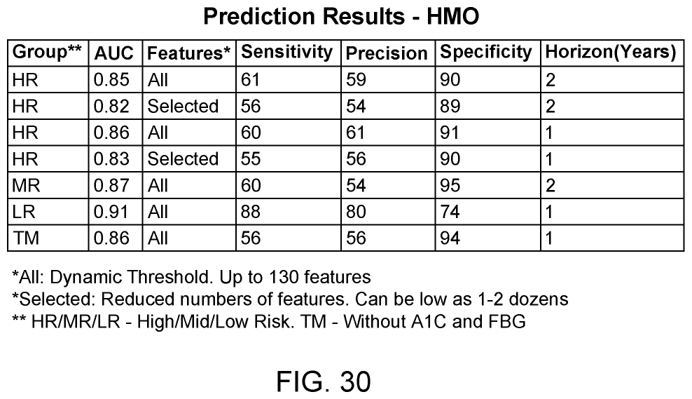

Example results for DT2 in one embodiment may be:
A. Training on an HMO dataset (e.g., of an Israeli HMO). Testing on orthogonal/different records of the HMO data set. Some possible combinations are shown in FIG. 30.
B. Training on a first HMO dataset (e.g., of an Israeli HMO). Blindly Testing on different (e.g., American) data set—i.e., training and testing are done with totally different data sets. Results Achieved: Area Under Curve of 0.81.

In accordance with an embodiment, the following is a list of example relevant conditions in which the systems and methods may provide recommendations for treatment and/or prevention:

Diabetes - Type 2
CVD
HTN
Pre-diabetes
Obesity
MSK
Rheumatoid Arthritis
OSA
NAFLD
Hypercholesterolemia
COPD
Osteoporosis
Depression
Breast cancer
Oncology
Diabetes - Type 1
IBD
CKD
HIV
Epilepsy
Gaucher (rare disease)
Gestational Diabetes
PCOS
Sedentary lifestyle
Over Weight

In accordance with an embodiment, Urine parameters may be utilized to provide feedback and information to the user. As an example, urine parameters may be set as follows:

### Normal Values are as Follows:

Color—Yellow (light/pale to dark/deep amber)
Clarity/turbidity—Clear or cloudy
pH—4.5-8
Specific gravity—1.005-1.025
Glucose—≤130 mg/d
Ketones—None
Nitrites—Negative
Leukocyte esterase—Negative
Bilirubin—Negative
Urobilirubin—Small amount (0.5-1 mg/dL)
Blood—≤3 RBCs
Protein—≤150 mg/d
RBCs—≤2 RBCs/hpf
WBCs—≤2-5 WBCs/hpf
Squamous epithelial cells—≤15-20 squamous epithelial cells/hpf
Casts—0-5 hyaline casts/lpf
Crystals—Occasionally
Bacteria—None
Yeast—None

As another example, in accordance with an embodiment, Stool data may include:
Type 1: Separate hard lumps, like nuts (difficult to pass and can be black)
Type 2: Sausage-shaped, but lumpy
Type 3: Like a sausage but with cracks on its surface (can be black)
Type 4: Like a sausage or snake, smooth and soft (average stool)
Type 5: Soft blobs with clear cut edges
Type 6: Fluffy pieces with ragged edges, a mushy stool (diarrhea)
Type 7: Watery, no solid pieces, entirely liquid (diarrhea)

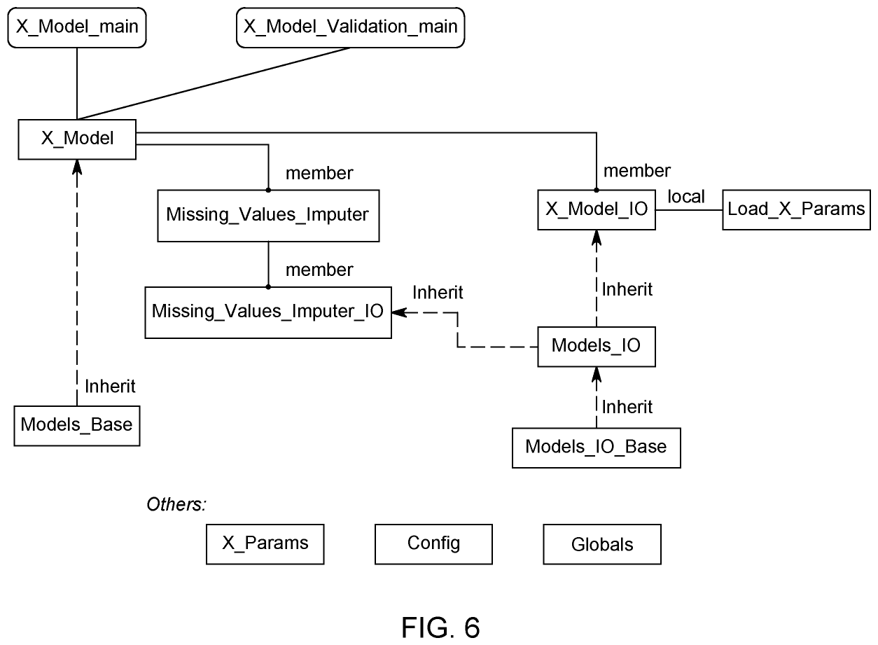

FIGS. 5 and 6 illustrate examples of code structure diagrams for models and ensembles, in accordance with embodiments herein. FIG. 5 shows the architecture of the processing unit as described in FIG. 3. As shown in the Figures, in accordance with an embodiment, the Infrastructure unit may include:
FileIO: CSV_IO-handle readqwrite toqfrom csv file
Logger-Handle all logging operations. Results are in Log Folder
Algo_DailyMessages_Queue-RL Generated Daily Messages Table-Helper Table. Messages are generated by the Algo units

In an embodiment, as shown in FIG. 6, the ModelsNEnsambles unit may include:
Models_Base-Abstract api for all algorithmic models operations
Models_IO_Base-Abstract api for all input/output models operations.
Models_IO-generic input/output models operations. The class contains input parameters and constants that are common among all algorithmic models. It includes data files names for all models and Imputer.
X_Model-specific implementation of X algorithmic model
Hinge_Model-implementation of the Non-linear Hinge model
Random_Forest_Model-skleanr implementation
Logistic_Regression_Model-skleanr implementation
X_Model_IO-IO for the X_Model. The class contains only the parameters of the specific model.
Missing_Values_Imputer-preprocessing class to treat missing values in data.
Model specific class (strategy value setting).
Missing_Values_Imputer_IO-IO class for Missing_Values_Imputer.
Load_X_Model_Params-loading parameters of X model from appropriate file.
X_Model_main-main function to run training&testing of model X for various sets.
X_Model_Validation_main-main function to test already trained model X on validation data
Validation_Params-specific class for Validation mode. It contains validation parameters for each Risk Group.
Ordered_Records-data assumptions & constants

In an embodiment, Others Unit(s) may include:
X_Params-object with all parameters for each model type, including Pivot_Params-parameters for various pivots models and LR_Params
Config
Global

Accordingly, it should and will be understood based on this disclosure that, as an AI-based fully automated solution, Sweetch enables scalability and business flexibility that may change the lives of tens of millions of people. Sweetch is a platform that includes both an individualized quantified risk prediction engine and a hyper-personalized intervention engine. Sweetch is a solution that continuously and automatically adapts itself to the patient's real-world behavior.

The disclosed Sweetch modular solution is applicable to and offered across several disease domains and population segments. This approach represents a significant advantage for pharma companies, providers, insurers, and employers as they all serve different patient populations. Further, the disclosed systems and methods provides feasibility and ease of implementation advantages; i.e., by being a fully automated solution, Sweetch enables easy and streamlined deployment processes at scale.

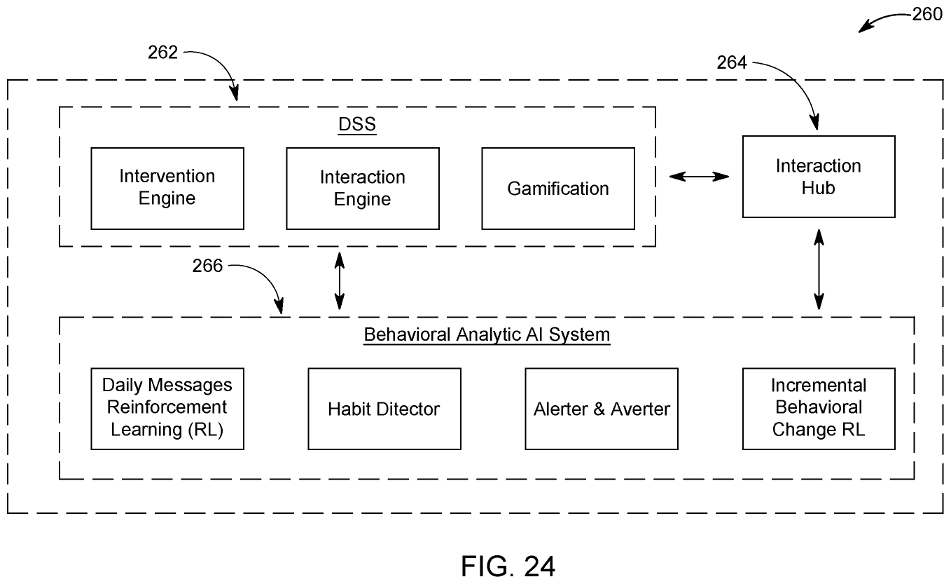

Moreover, as described herein, several capabilities are provided in the disclosed platform, including:
Just-in-Time Adaptive Intervention (JITAI)—This technology was built with the complexity of chronic disease patients' behavior in mind. The idea of capturing the user's right moment requires orchestrating behavioral change in a way that will increase the likelihood of action on most fronts (winning the “game” rather than winning a “round”). This includes improving disease management-medication adherence, adherence to follow-up and treatment protocols, patient-reported outcomes, and patient education, alongside the adoption of healthier life habits-physical activity, weight management, and diet. The JITAI mechanism is based on a combination of advanced algorithms, among them reinforcement learning, neural networks, predictors and more. It includes a dedicated feedback loop for adapting and adjusting the program according to information received from the patient. The system studies behavior patterns and incorporates them into the proposed Intervention mechanism.
Prediction of Personalized risk quantification—the platform includes of advanced ML algorithms for detecting and predict patient's health condition and their risk of developing a chronic disease. For each patient, a unique Index score is built, that reflects the personal level of risk in the context of his/her disease. The company has developed an innovative model, while identifying thousands of markers (personal, behavioral, physical etc.) that have a significant impact on the degree of patient's risk. These include both familiar and clear features and complex (Compound/Complex) traits such as correlation between weight data, lab test, medications and vital signs, all are tested over time, and across population groups with similar characteristics. This development uses a variety of learning algorithms and decision rules which are integrated together to allow for better and more accurate prediction. This activity requires proper definition of the algorithms, understanding the various parameters and establishing an optimal architecture for calculation.
Hyper Personalized approach—By implementing AI-powered Behavioral Intelligence Engine, Sweetch analyze substantial data originating from a patient, to create deep understanding of the patient's behavior, motivation and challenges. Accordingly, Sweetch offers customized and adaptive training program, providing an on-going comprehensive view of the patients' behavior, outcomes, measures and treatment adherence patterns. This allows to undergo dynamic adaptation and intervene only when intervention/support is truly needed. Sweetch predicts patients' behavior in different contexts, to promotes healthy lifestyle, disease management and weight control. This technology enables real-time, individualized, and dynamic support that empowers sustainable health behavior change in a highly cost-efficient and scalable way.
Chronic diseases management and implicate medication adherence—The platform includes methods and decision-making protocols to enable diseases management and Medication Adherence. Features, parameters and logics are considered such as the type of treatment, type of medication, the patient's behavior towards his disease, dosage, treatment's restrictions and more. Algorithms sync all those variables, while considering the overall state of the patient at a specific time frame and execute an optimal intervention. This allows improving disease management-medication adherence, adherence to follow-up and treatment protocols, alongside the adoption of healthier life habits (physical activity, weight management, and diet).
Leverage understanding of patient's behaviors-Sweetch use additional approaches and tools to ensure the compliance and adherence of different people with different personalities. Accordingly, methods and techniques provide a deep understanding of patient's behaviors. This includes:
Habits Detector and Alerter & Averter AI (A&A AI) units (shown and described with reference to FIG. 24), that automatically detect recurring habits or patterns of future behaviors and by implementing ML techniques (NLP prediction algorithms, Clustering algorithms, Similarity/dissimilarity algorithms etc.), understand their context and implication on the patient's health condition. The A&A AI unit is capable of providing a-priori alerts by recommending alternative actions to take genetic optimization. These methods perform, among other methods, mutation, crossover and selection in order to find by “natural selection” suitable series sampling that will be used to predict incoming events needed to be alerted.

Methods that define a digital Persona by creating “Blue Prints”, which are a set of unique traits and features that define the user holistically. These unique per-user (Table 1) and per typical day (Table 2) digital “Blue Prints” enable Sweetch to capture the core of each user and then classify him/her and categorize him/her. All personas as well as the “Blue Prints” are algorithmically defined and optimized by different aspects of the user's daily life, habits and routines, and internal and external traits.

TABLE 1
Patient “Blue Print” example

| Pilar | Trait | Value & Description |
|---|---|---|
| B | BMI | 31.8 |
| | BMI stability | 30.9-32.1 @ last 90 days |
| M | Commuting | Mostly by Train |
| | | Average daily 90 min |
| | Shopping | Spend 90 min a week at supermarket mostly |
| | | at Thursday night |
| | Restaurants | Every Tuesday night |
| A | Walking | Walks to/from office: 12 min |
| | Running | Run at least twice a week |
| | Activity | Mostly at evening |
| | Patterns | Weekend walk at the open country |
| D | Diner | Like salad bar |
| | | Mostly vegetarian |
| | Fasting | Every Monday. No Breakfast reported |
| Medicine | Type | Saxenda for Obesity |
| | Usage pattern | Seldom miss. Sharp on time |

TABLE 2
Typical Day “Blue Print” example

| Time | Act | Place/Duration |
|---|---|---|
| 7:00 | Wake up | Slept 7 hours |
| 9:03 | Arrived Office | Driving 90 min |
| 9:10 | Medicine Intake | Saxenda for Obesity |
| 12:20 | Dinner | Salad Bar. 30 Min |
| 19:40 | Arrived Home | Driving 50 min |
| 20:30 | Gym | 90 min |
| 23:00 | Smartphone usage | 20 min |
| 24:00 | Sleep start | |

A sophisticated AI engine that handles incremental goals behavioral change—development of an engine that continuously revisits the patient's goals set and may change them based on real-life data, directing the user to have a course of wins that leads to sustainable behavioral change. This is an open problem in the field of algorithmic reinforcement learning, thus represents a novel approach. Sweetch has built a deep reinforcement learning network to generate multiple goals arena using sparse rewards and mechanism such as attention and intrinsic reward known as Curiosity—these are quite new additions to the Deep Learning pleura from the very recent years.
Advanced AI-based interaction aims-means that optimally deliver messages and/or content to the user, while achieving maximum responsiveness. Interactive tools (PROMs, Conversional bot, Nutrition board etc.) interact with the user in different methods and levels. Sweetch analyzes and identifies the data from the patient, and accordingly tailors the relevant interaction (by push notification, in-app pop-up, in dedicated areas in the app, PROM, Bot etc.) in the exact time, with maximum relevance to each patient. Each tool is dynamically tailored to the user's response, presented in a personalized manner to help patients through their journey in different disease states by easily reporting and/or asking questions and/or just tracking automatically.

Further details regarding the disclosed technology are now described.

### Prediction Platform:

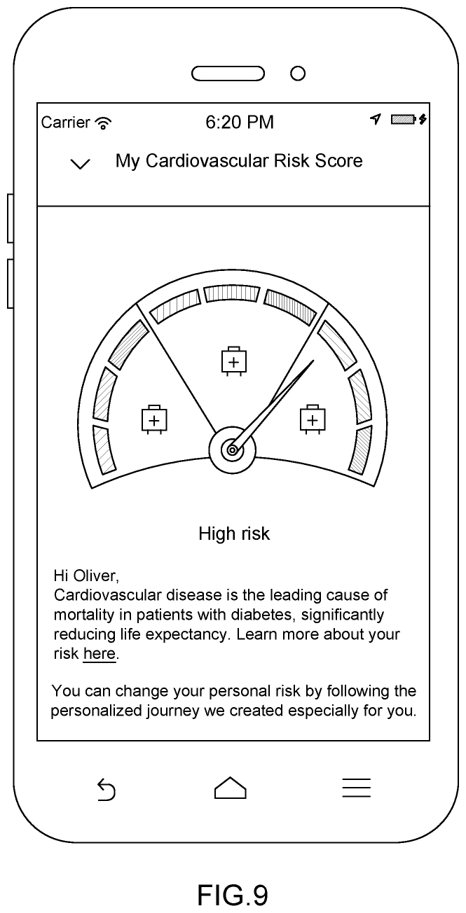

The disclosed systems and methods provides a platform of machine learning algorithms that enable calculation of an individual quantified risk in accordance with his disease. In embodiments, the prediction platform is part of (e.g., a subsystem) of the disclosed system. In other embodiments, the prediction platform may be provided as a separate, stand-alone system that communicates with the disclosed (intervention) system. The algorithms take into account the patient's medical history and behavior over time, the disease current state and the required treatment. In an embodiment, the prediction platform is configured to predict different scores of conversion to different conditions (such as those in the earlier described list of example relevant conditions). Based on these, the platform predicts risk ahead in specific timeframe. Predictions may be communicated to the user (e.g., such as shown and described with reference to FIG. 9) and/or use as part of the user's “Blueprint” as described throughout the disclosure. The disclosed predictive platform includes the construction of features that capture informative predictive patterns, in accordance with embodiments. In an embodiment, the algorithm may be built of three different modules: feature engineering, classification, and risk estimation. In addition, to increase accuracy and validity, the disclosed systems and methods may supply a confidence value for the predicted risk. The models employ predictive features that include measurements from the medical records as well as the following features:
Basic set—Common features are already known from the literature and relate to the clinical outcomes.
Novel features—The platform identifies features which had no known correlation to clinical outcomes and are found to improve ranking accuracy.
Constructed features—Novel predictive features that are derivatives of information from the medical records.

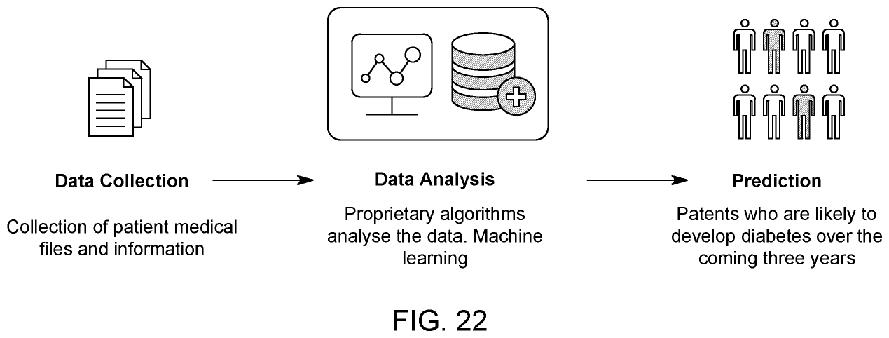

By implementing data-driven machine learning and constructing set of models with a learning mechanism that focus on previous errors in order to improve and construct better models, the disclosed systems and methods accommodates heterogeneous predictive patterns, and provides better predictions for highly heterogeneous patient population. The predictive modelling produces a vast number of different predictive models, incorporating native predictive features, novel features, and constructed features. FIG. 22 (described later) generally illustrates an example of Sweetch prediction flow in accordance with an embodiment.

The platform also includes mechanisms to effectively combine inputs from multiple models to yield robust and accurate predictions. Differently from most modeling initiatives published so far, the disclosed approach combines data-driven techniques and domain knowledge to identify informative predictors and do it in an ongoing manner. This personalized risk stratification provides better patient compliance and adherence as well as for triggering a more aggressive treatment approach in patients who are found to be at higher risk. A quantified personal risk serves as a strong driver for behavioral change and allows a personalized intervention that results in better health outcomes.

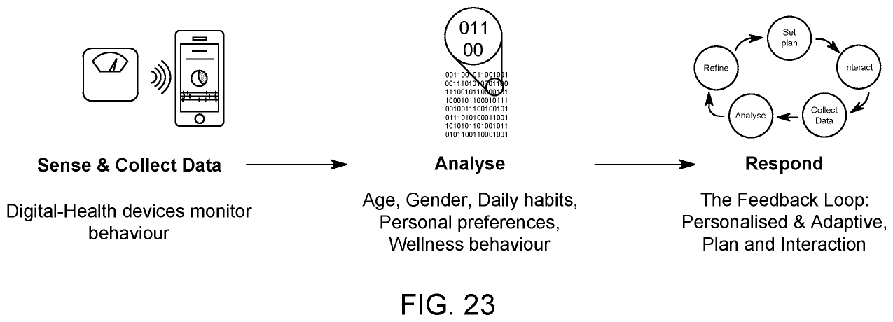

Some benefits of the disclosed intervention platform include, for example, meeting the goal to achieve sustainable healthy habits through behavioral change; guiding the user towards improvement with incrementally changing goals, for example: Increased Walking, Eating Smaller Portions; and encouraging adherence to treatment protocols. General details regarding the intervention platform are shown and described with reference to FIG. 23.

The disclosed platform is composed of two complementary components, which are described in greater detail below and throughout this disclosure: Personalized risk quantification-Prediction of individual's risk for developing chronic disease (for example, converting from pre-diabetes to diabetes) and for developing comorbidities and long-term complications; and Hyper-Personalized intervention-Significantly increase of patients' adherence to essential disease management and health promotion recommendations in a unique, AI-based, highly personalized, and scalable way. In accordance with embodiments described herethroughout, the following are exemplary features that may be provided as part of an intervention system of this disclosure.

In accordance with an embodiment, the intervention system includes Relevant Domains for Intervention or Management of:
Physical activity such as: Walking, Running, Swimming, Yoga, any exercises taken whatsoever
Weight
Diet and nutrition
Sleep
Mood
Addition
Medication-taking/supervising
Healthy habits
Treatment protocols
Connected devices
Promotions and questionnaires
Gamifications
Social
Epidemics tracking
Glucose monitoring such as blood glucose monitoring (BGM) and continuous glucose monitoring (CGM)

In accordance with an embodiment, the systems and methods are designed to aid in prevention of any of the aforementioned conditions previously and/or subsequently described.

Input to the system may include any of the features noted with regards to the Data Input & Analysis previously noted above. In accordance with an embodiment, input may be one or more of the following (in addition to or alternative to the previously noted system inputs):
Demographics data, such as: Age, gender, occupation, socio-demographic status, address, race, existing/inherited diseases or tendencies to diseases

Any information collected by questioners from the app or during or before onboarding (such information being relayed to the systems 100, 350, etc. herein). Questioners may include but are not limited to user Behavioral state.
Physical Activity data such as the amount of PA performed: duration, time of the day, intensity, in which physical places etc.
Geo location data: physical locations, visiting places, Connection Wi-Fi or any other cellular nets
Weight Data-performed by the user using a digital/analog scales
Connected devices data-vital signs, BP, Breath, CGM/BGM data-either automatic or manual, digital scales, wearables
Meals reporting-Amount, Healthiness, Time, different ingredients, drinks—amount, size & type, pictures, audio reporting
Medications: type, Amount, frequency, time of the day, location taking
Sleep Data such as sleep duration & hours
Mood data such as “emotional feeling”, feeling depressed y/n etc.
Calendar information
Mobile device activity and responses
Fecal Data—e.g., questions such as what patient's fecal matter looks like, consistency, etc.
Health habits-Determine your health habits and/or your own stated goals
Gamification Data-Setting challenges and competitions and perceive user response

In accordance with an embodiment, input data may be any one or more of the following:
Sparse in values or abundant
Discrete or continues
Over time or single event
Automatic or manual
Through specific questions or as an open text
In audio form or clicking or entering text or taking pictures
Through any sensor that might be exist in any smartphone, among these: GPS, accelerometer, gyroscope, light sensor, camera, retina, etc.
be categorial, ordinal, integers, floats-being a scalars or vectors or matrixes
Single population/country or not

### In Accordance with an Embodiment, Behavioral Models May Include

TTM—Transtheoretical models
Any set of questions that try to assess user behavior, inner self/positions such as willingness to change etc.

In accordance with an embodiment, the system inner mechanism of change may be one or more of the following:
Alerters
Averters
Programs-include setting single or multiple goals per domain or cross-domain
Incremental changes per domain or cross domain for single or multiple goal

Further, in an embodiment, the inner mechanism of change may be based on one or more of: adaptive algorithms, set of rules, static/dynamic features.

Inner representations of the user (blueprints) may be based on Persona traits, according to an embodiment. These persona traits may include different features for each & every described domain or can be cross domain features, according to embodiments.

In accordance with an embodiment, there are several usage flows that may be implemented based on this disclosure. In an embodiment, one or more of the following exemplary flows may be utilized:

### Physical Activity (PA)—

i) User performs PA on a daily basis: Walking, Running, Swimming etc.
ii) System measures user's daily number of minutes or the user reports manually
iii) On a weekly basis—the system set a weekly & daily goal
iv) The user is asked to reach this daily & weekly goal
v) The system might filter non-significant chunk of walking and/or chunk of steps that are too short
vi) The system will adapt the weekly goal on a weekly basis, the daily goal on the
daily basis
vii) The system will dynamically adapt weekly and daily goals based on the user behavior
viii) The system will send to the user specific interactions, such as at least one of: a specific message, reminders, encouragement, reward(s), badge(s), competition(s), an alert, an averter, a trigger, a recommendation, content recommendation, and interact with the user in any other form in order to guide him towards reaching his goals

### Weight Management—

i) User should report his weight on a personally scheduled reported basis (e.g., weekly, daily)
ii) On a weekly/reported basis—the system will show him his current and former weight, as well as his trend of change
iii) The user is asked to reach a goal of reducing 5% to 7% from his base weight
iv) The system might send reminders to the user, urging user to weight himself/herself
v) The system may set a weight reduction plan to the user, urging the user to reduce weight based on a multiple weeks plan with a modest reduction in weight
vi) The system may adapt the Weight goal on a weekly/other than weekly basis

### Meals Reporting—

i) User should report meals on a daily basis
ii) On each reported event—the system will show the user inferences that are based on the user's personal journey before the actual reporting
iii) On each reported event—the system will show the user inferences, after reporting, that are based on the user's personal journey
iv) On a daily/other predefined set of time, the system will choose a set of meals-related incremental goals. These incremental goals will be sent to the user, encouraging the user to achieve these goals. Upon achieving these goals—the user will receive a badge, i.e., to celebrate success.
v) The System will use an adaptive algorithm to determine the perceive/most suitable goals to achieve

Of course, the above flows, features, and data are exemplary only and not intended to be limiting. Further details regarding flow, features, data, engines/modules, and devices associated with the disclosed system and method are described below and may be used in addition to or as an alternative to any of the previously described elements.

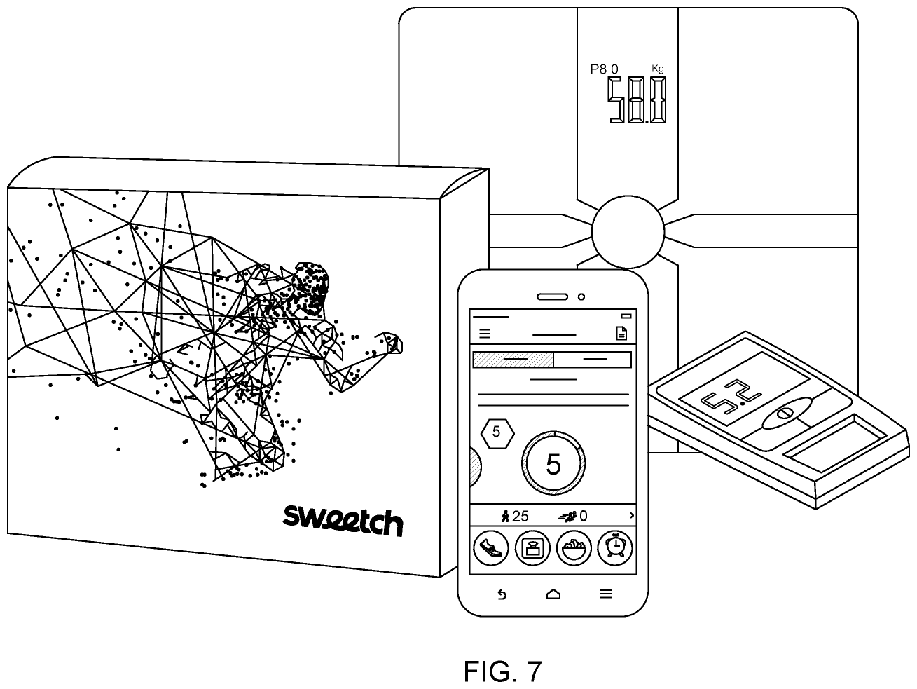

Turning to FIG. 7, an example of a Sweetch Intervention Package, in accordance with an embodiment, is shown. Such a package includes, for example, connected device(s) and an app for downloading and access via a mobile device.

Sweetch's approach combines remote patient monitoring via connected devices (i.e. glucose meter, digital scale, blood pressure monitors etc.), advanced data science technologies (AI, machine learning, signal processing, statistics and other deep learning technologies), established behavioral science approaches, and persuasive technologies inspired by mobile gaming and social empowerment worlds. Accordingly, the disclosed systems and methods are designed to provide personalized risk quantification and hyper-personalized intervention to a user. In particular, Sweetch serves as a beyond-the-pill component for fully automated disease management optimization for patients with pre-diabetes, diabetes, hypertension and other chronic conditions. Designed to work in real life, Sweetch's AI-powered Behavioral Intelligence Engine (BI Server) converts millions of data points originating from a patient's smartphone and other connected devices into contextual, hyper-personalized, just-in-time, just-in-place, recommendations. Through real-time analysis of the patient's current context and past behaviors, Sweetch's algorithms continuously optimize the recommendations that each patient receives in a way that increases the likelihood of action.

The disclosed systems and methods may be implemented on iOS and Android platforms. Sweetch's Just-in-Time Adaptive Intervention (JITAI) technology improves patients' disease management by increasing their adherence to medication and treatment protocols, capturing their Patient Reported Outcome Measures (PROMS) and allowing their HCPs to monitor and act, when needed, in real-time. At the same time, Sweetch's JITAI advances patients' adoption of healthier life habits such as physical activity, weight management, diet and more. Similar to how Precision Medicine is able to tailor treatments to an individual patient, the JITAI technology continuously and dynamically tailors recommendations to the patient's personality, daily routines and habits, motivations, care journeys, and challenges. In accordance with an embodiment, data is sensed and collected from multiple sources, as described above (and later) with regards to systems 100, 3500. Collection of data and analysis of user's context is performed in real-time. Further, the system is designed to engage and optimize personal (user-specific) recommendations to the user (or patient) via utilizing an AI engine, so that a personalized, dynamic, patient experience is presented. Data is collected, analyzed, and refined, and a plan is set to interact with the user. Further, a medical team management console may be provided as part of the technology. The system is configured to provide machine learning predictions, as well as preparing and configuring a risk index.

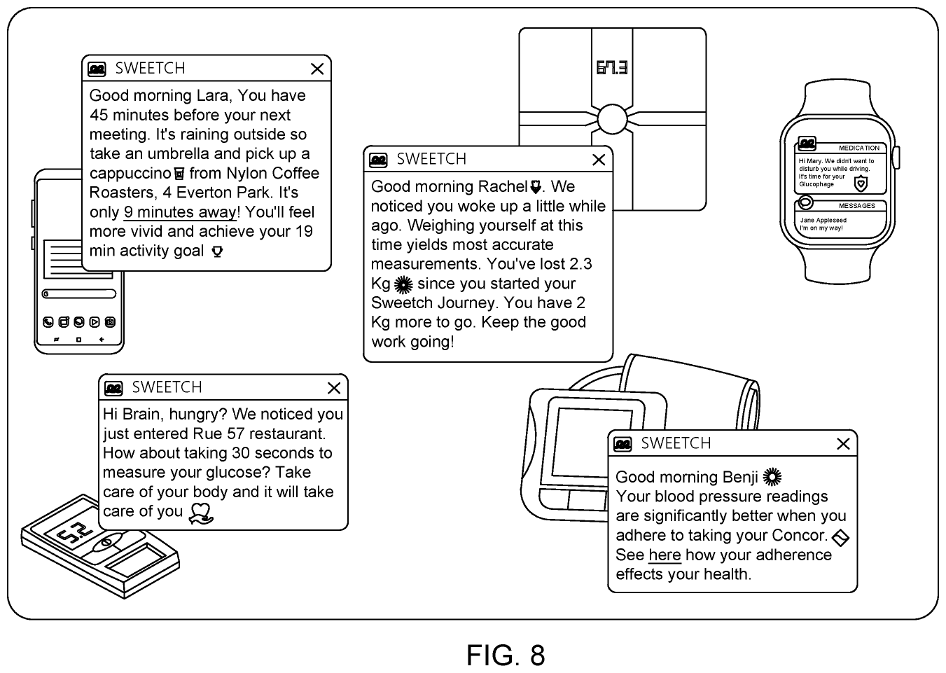

FIG. 8 illustrates an example of personalized messaging that may be presented to the user, in accordance with an embodiment. As described in greater detail with regards to FIGS. 9-15, for example, messages presented to the user may be personalized based on the habits, conditions, and lifestyle features that are being monitored such that the system tailors output messages for the user. FIG. 9 illustrates an example of a display showing an exemplary output to a user in accordance with an embodiment. In this example, a condition is monitored for the user and a scale and message relating to the user's risk (e.g., cardiovascular condition) is presented as part of the prediction component. In particular, FIG. 9 shows an example of a prediction score that the system provides for conversion or complication to a metabolic disease (heart disease) for a particular user. Accordingly, the system (including the prediction platform) is/are designed to, in accordance with embodiments, assist the user via communicating a predicted risk ahead in a particular/specific timeframe and/or the risk prediction as part of the user's “Blueprint”.

Accordingly, the system provides one dynamic platform for multiple conditions. The disclosed systems and methods are applicable and may be customized to different disease domains. For example, the platform can be deployed in at least the following five disease verticals, in accordance with an embodiment:
Cardiovascular and Metabolic Areas-Diabetes, Pre-diabetes, Obesity,
Hypertension, Coronary Artery Disease and Congestive Heart Failure;
Oncology-Breast Cancer and Prostate Cancer;
Rheumatology-Rheumatoid Arteritis
Gastroenterology-Inflammatory Bowel Disease
Orthopedics-rehabilitation after hip/knee replacements
Unique Data Asset

Combined with clinical outcomes, Sweetch comprehensive insight of patients' behavior, reported outcomes, biometric measures and treatment adherence patterns, creates a unique data asset that doesn't exist today. Such data asset has significant implications on chronic disease management.

### Sweetch Solution Components

As mentioned throughout this disclosure, the disclosed systems and methods provide both Personalized risk quantification and Hyper-Personalized intervention. In an embodiment, Remote Patient Monitoring (RPM) and a Management Dashboard is also provided.

### 1. Personalized Risk Quantification:

Unlike existing diabetes risk calculators which present a vague risk estimation, i.e., low/medium/high, Sweetch's machine learning algorithm enables calculation of an individual's quantified risk for developing chronic disease and associated complications in specific timeframe. This personalized risk stratification provides better patient compliance and adherence as well as for triggering a more aggressive treatment approach in patients who are found to be at higher risk. Beyond being a highly-accurate screening tool for physicians, medical teams, payers and providers, knowing one's quantified personal risk serves as a strong driver for behavioral change and will allow a personalized intervention that will result in better health outcomes.

### 2. Hyper-Personalized Intervention:

Sweetch monitors and analyzes personal, environmental and behavioral digital biomarkers. The platform automatically learns various aspects of the user's life habits by applying advanced analytics to millions of data points extracted from the patient's smartphone sensors. At the same time, the platform collects disease-specific data such as PROMs and adherence to treatment and medication protocols. This results in contextual, real-time, personalized, just-in-time, just-in-place, recommendations that guide the user towards achieving the improved heath goals with an emphasis on fitting into the patient's daily life.

By applying predictive analytics, Sweetch continuously optimizes the user's goals and messages, so they will be in the context, time, place, and tone-of-voice that increase probability of action. Data contextualization (e.g., user is at home, user is commuting to work, user's favorite coffee shop etc.) is done automatically, by continuous analysis of data streams.

Sweetch essentially predicts patients' behavior in different contexts and analyses what would be the recommendation that would most probably trigger the patient to take action. Sweetch offers holistic health promotion and chronic disease management optimization through increasing patient adherence across several aspects.

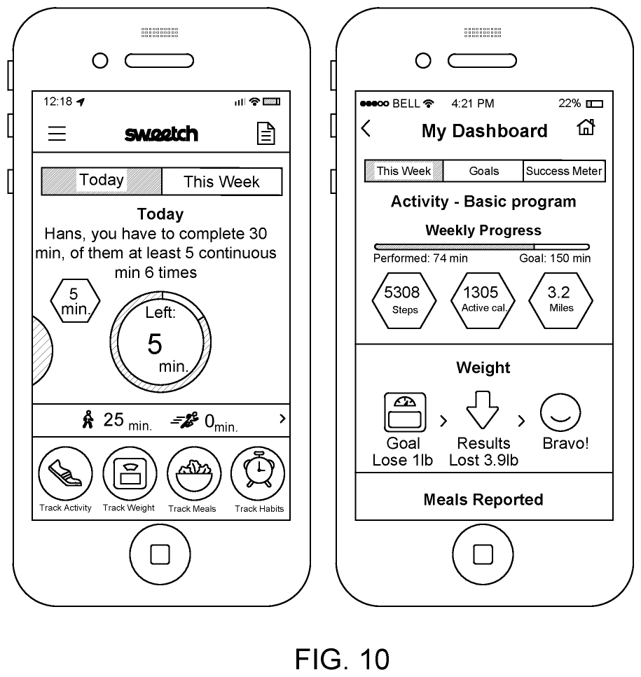

In accordance with an embodiment, one aspect includes activity management. FIG. 10 illustrates examples of display screens showing features related to Activity Management for a user in accordance with an embodiment. Activity may be monitored, for example, using the millions of digital “biomarkers” data points collected from the user's mobile device, including demographics, geo-location, schedule, actual activity patterns, outside weather and more, to turn the usual, non-personalized, and non-engaging recommendations of “walk 10,000 steps/for 30 minutes a day” into personal, contextual, real-time, recommendations like, as shown in FIG. 8: “Good morning Gary, you have 45 minutes before your next meeting. It's raining outside so take an umbrella and pick up a cappuccino from Nylon Coffee Roasters, 4 Everton Park. It's only 9 minutes away! You'll feel more vivid and achieve your 19 min activity goal”.

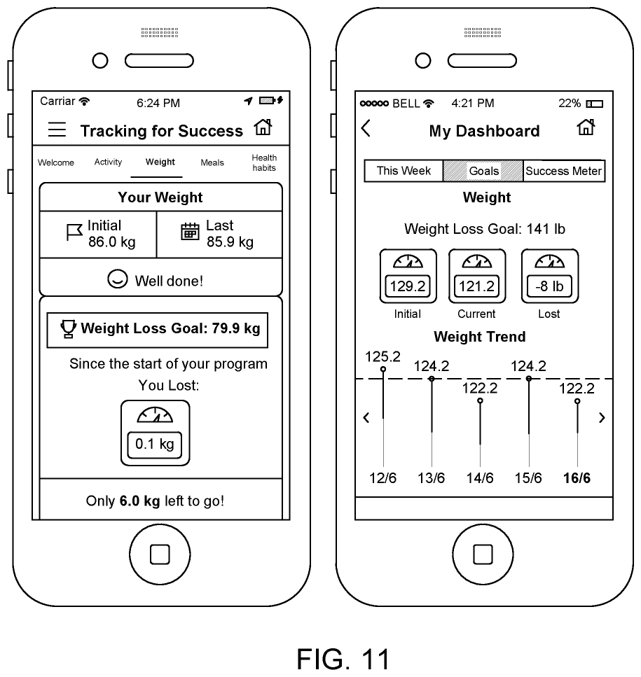

In accordance with an embodiment, one aspect includes Weight management. FIG. 11 illustrates examples of display screens showing features related to Weight management in accordance with an embodiment. By monitoring a user's weight, healthy weight management is promoted. According to an embodiment, Sweetch gathers a users' weight data via manual input or via a connected scale device through the application and sends a user contextual and personalized push notification, in app pop-ups, in-app messages, rewards, gamification related messages and any other interaction at the right time, context, content, and sentiment to try to improve a users' mindfulness about their weight and weight tracking. If required, e.g., based on a users' weight and BMI, the system dynamically sets, reassesses and optimizes a users' weight goals based on the users' daily behavior and their adherence to certain weight-related interactions, according to embodiments herein. Sweetch may also provide and/or integrate to Bluetooth digital scales and collect weight data seamlessly according to an embodiment.

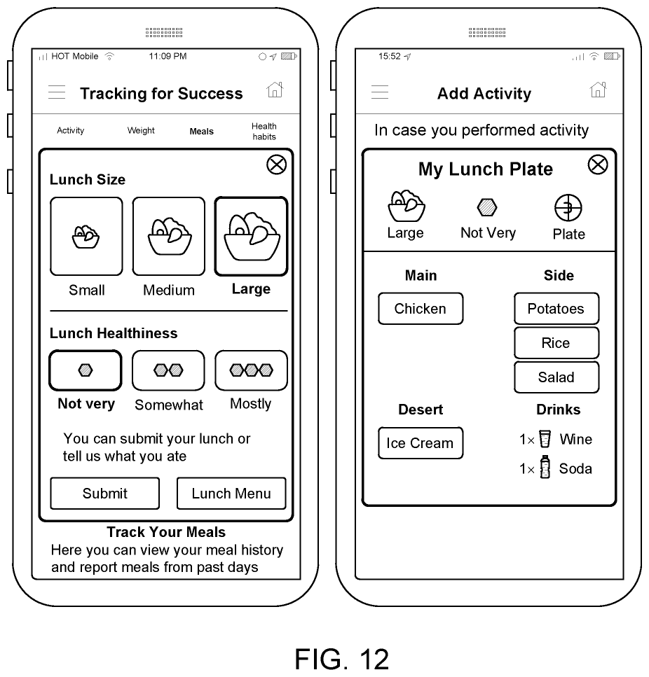

In accordance with an embodiment, one aspect includes Diet and Nutrition. FIG. 12 illustrates examples of display screens showing features related to Diet and Nutrition in accordance with an embodiment, which may be used to target long-term engagement. Such an approach by the disclosed system and method promotes meaningful diet related reporting focusing on dietary awareness rather than on “obsessive” calorie counting. By focusing on meals size and healthiness with the combination of the user weight and activity, the disclosed system and method is able to provide a deep understanding of the user perception and enable Sweetch to direct the user to adhere to healthy diet and nutrition habits. Nonetheless, for patients who are looking to track the details of their meals, Sweetch allows reporting of meal items according to embodiments herein. This information may be used to calculate the different food groups, the ingredients and the calorie value, for example. To complement the diet and nutrition, in accordance with embodiments, Sweetch also enables drinks consumption reporting that tracks sugary soda, alcohol and water.

Further, the disclosed system also promotes adherence to additional healthy lifestyle habits defined by the user. These may include, among others, reducing stress, improving sleep hygiene, and increasing water intake. Sweetch assesses health habit goals that are manually inputted through the application drives users to improve their ability to achieve these goals based on contextual push notifications sent at the right timing and place.

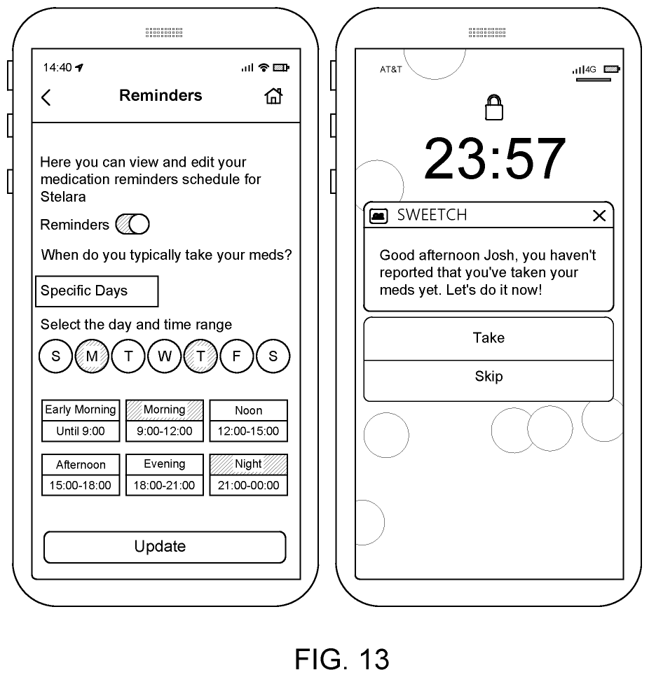

In accordance with an embodiment, one aspect includes Medication Management. FIG. 13 illustrates examples of display screens showing features related to Medication Management in accordance with an embodiment. In an embodiment, such may include treatment protocols and medications; Sweetch enables patients to define their treatment protocols and medications schedule from within the app, and/or receive it automatically from Sweetch management dashboard or the EHR. Sweetch promotes and reminds patients to follow their treatment and medication protocols, while taking into consideration the patient's life habits, availability and likelihood to act.

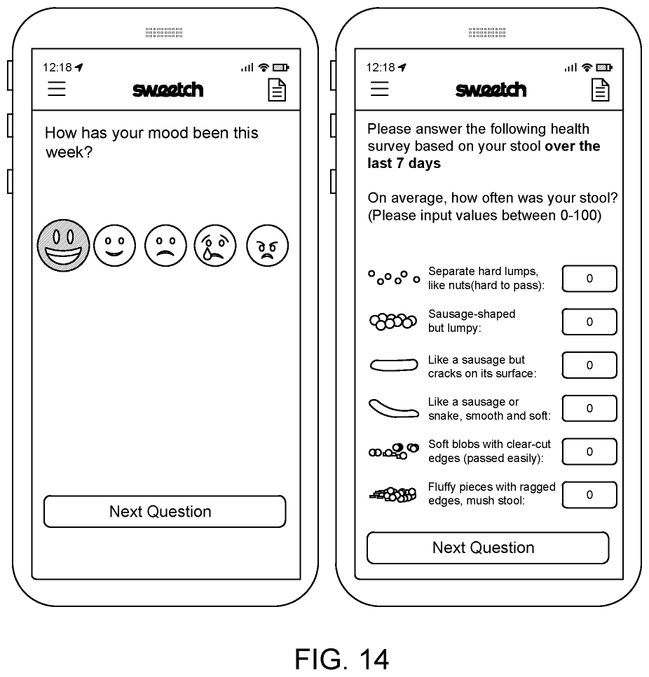

In accordance with an embodiment, one aspect includes Sweetch PRO Module. FIG. 14 illustrates examples of display screens showing features related to Sweetch PRO Module in accordance with an embodiment. Patient Reported Outcome Measures (PROMs) refers to relevant prompts for patient subjective assessments (Patient Reported Outcomes—PROs and questionnaires) including health survey, pain scores, mood scores and fatigue levels, which may be utilized and provided in accordance with embodiments herein. These PROs may be defined by the care coordination team, for example, and sent to each patient at the right time and in the right context to increase the probability of adherence for completion. Timing and content of questions may vary based on their answers while their HCPs are being informed about their condition in real-time.

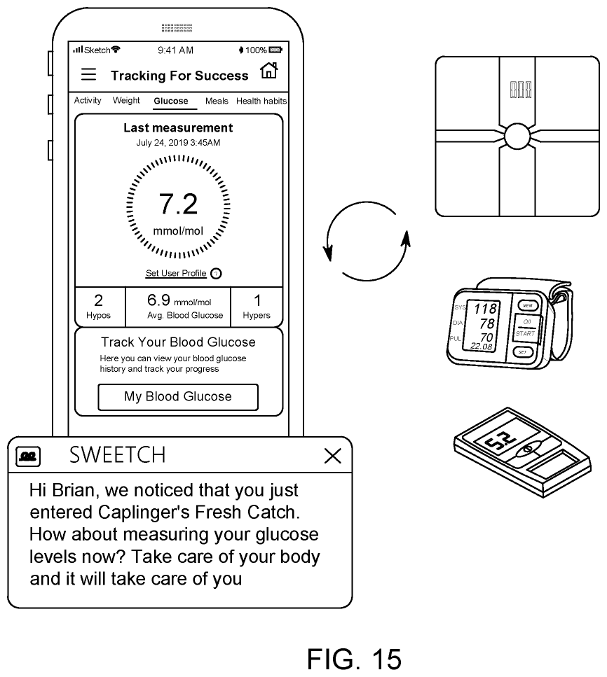

In accordance with an embodiment, one aspect includes a Connected device module. FIG. 15 illustrates an example of a connected device module of a Sweetch Intervention Package in accordance with an embodiment, which allows for remote monitoring (of any number of connected devices). In an embodiment, Sweetch includes a component that enables the app to connect to any connected device that has a direct API. Those, among others include, but are not limited to, BGM, CGM, scales, heart rate monitor, thermometer, smart watches, etc. The usage of the connected device may be defined by the patient or by the HCP. In an embodiment, Sweetch is configured to make sure to remind the patient/user to use those, based on the treatment protocol, daily routine, and availability. For example: personalize glucose measurement notifications in a way they fit the patient daily life and schedule. Options also include, recommending a glucose measurement when the patient leaves a restaurant or after physical activity. In an embodiment, such notifications will not appear when it is impossible for the patient to act, for example, when he/she is driving, in a middle of a meeting, or busy in any other activity.

### 3. Remote Patient Monitoring (RPM) and Management Dashboard

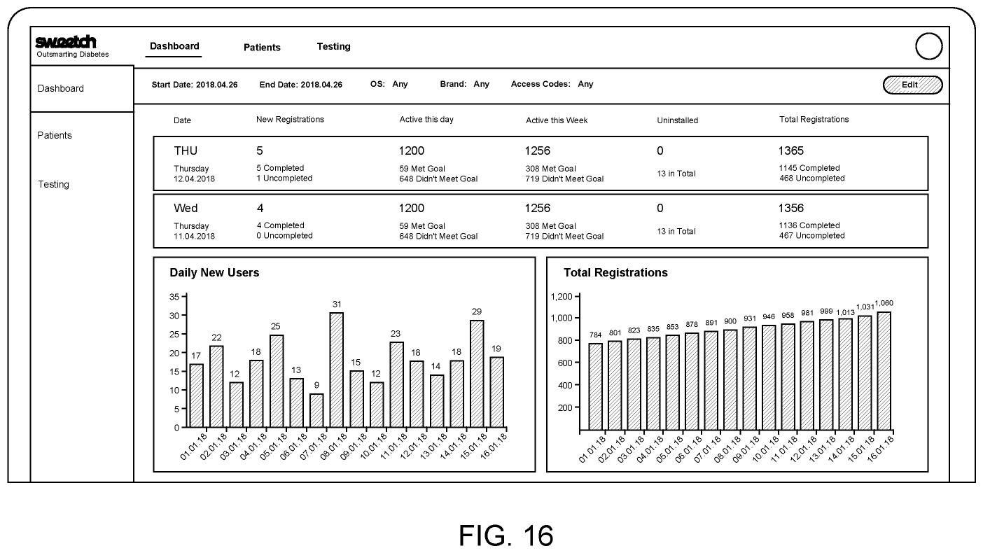

FIG. 16 illustrates an example of a Sweetch management dashboard in accordance with an embodiment. For care providers, healthcare organizations and Patient Support Programs (PSP), Sweetch offers a web-based RPM via a management dashboard that enables both population and individual views (HIPAA and GDPR restrictions apply-based on client's specific regulations). In an embodiment, Sweetch provides red flagging filtering of patients based on engagement and clinical outcomes. This way care providers and PSPs may move from a just-in-case interaction to individualized and efficient just-in-time interaction-identify at-risk patients to provide proactive outreach to those who require further attention. This approach strengthens physician-patient relationship, improves measurable clinical outcomes, and increases patient satisfaction. Sweetch may enable seamless launch of physician-patient communication, including digital clinic visits via video calls.

In addition other modules may be provided, including, but not limited to, education modules, gamification modules, social support modules, patient dashboard and data, and epidemic modules.

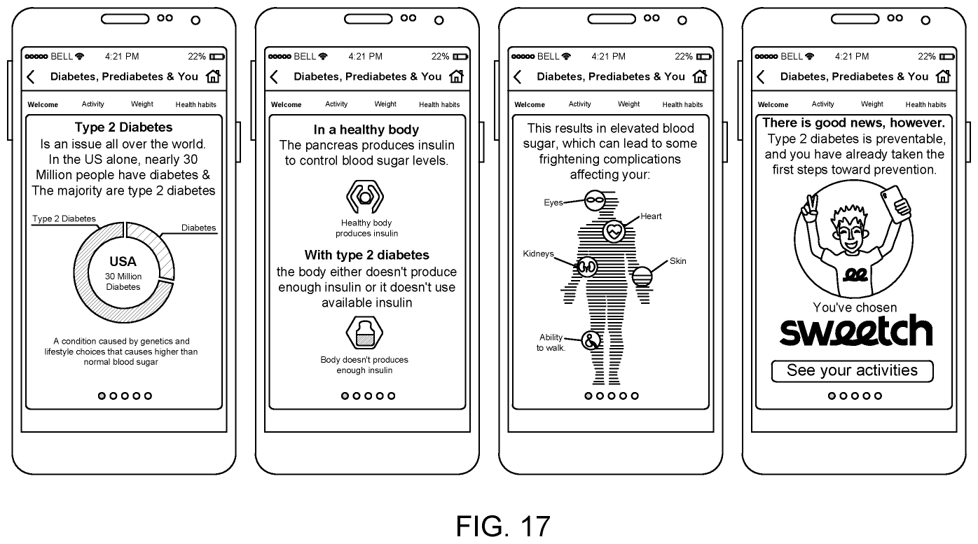

FIG. 17 illustrates an example of educational materials in display screen samples in accordance with an embodiment. In an embodiment, Sweetch creates a set of interactive and mobile-friendly lessons aimed to raise understanding and awareness to chronic disease and their complications with special emphasis on the ways to gain better control on the diseases and their consequences. For example, a company designed a Diabetes Prevention education content based on the Center for Disease Control (CDC) Diabetes Prevention Program (DPP) curriculum may be presented. In an embodiment, educational modules are adapted to match the exact client's requests and emphasis.

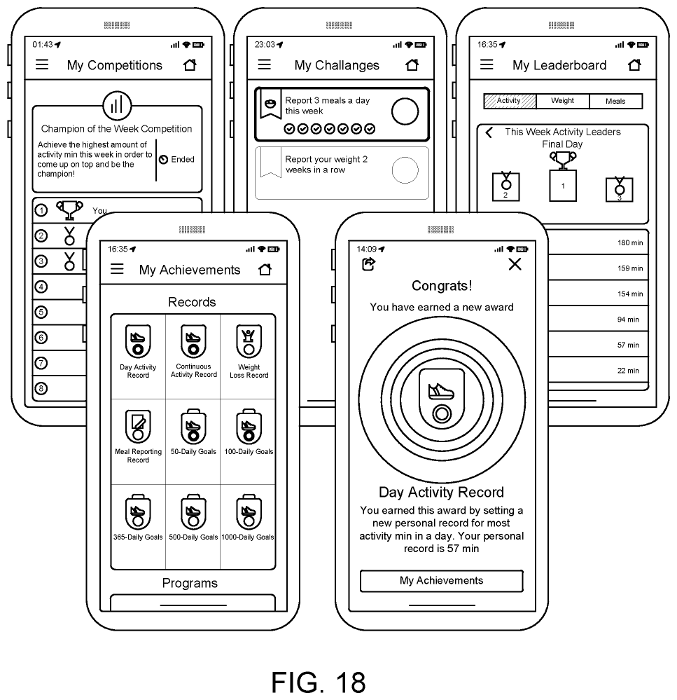

FIG. 18 illustrates an example of gamification features on display screens in accordance with an embodiment. In an embodiment, to ensure long term engagement and a more exciting patient journey, Sweetch may include a variety of gamification tools:
Competitions—personal competitions which are based on the patient's personal progress in the program to encourage him on areas where he struggles with.
Leaderboards—represents the patient's success in comparison to his community and peers which is meaningful in increasing motivation.
Challenges—personal challenges focusing on specific areas where Sweetch AI identified as required additional attention.
Achievements-personal badges achieved by the patient.
Rewards: Sweetch includes the infrastructure for integrating gamification-based rewards including real-world rewards, healthy points, reduced premiums etc.

In an embodiment, a social support module may be provided. Such social support may include, but is not limited to:
Supporter—to allow better personalized support system, patients can define a personal supporter from their friends & family to follow up on their progress and provide additional support in their journey.

Matched Social Reinforcement Network-Based on demographics, life habits and actual engagement patterns, patients are matched with an online peer group and a sponsor for added encouragement and accountability.

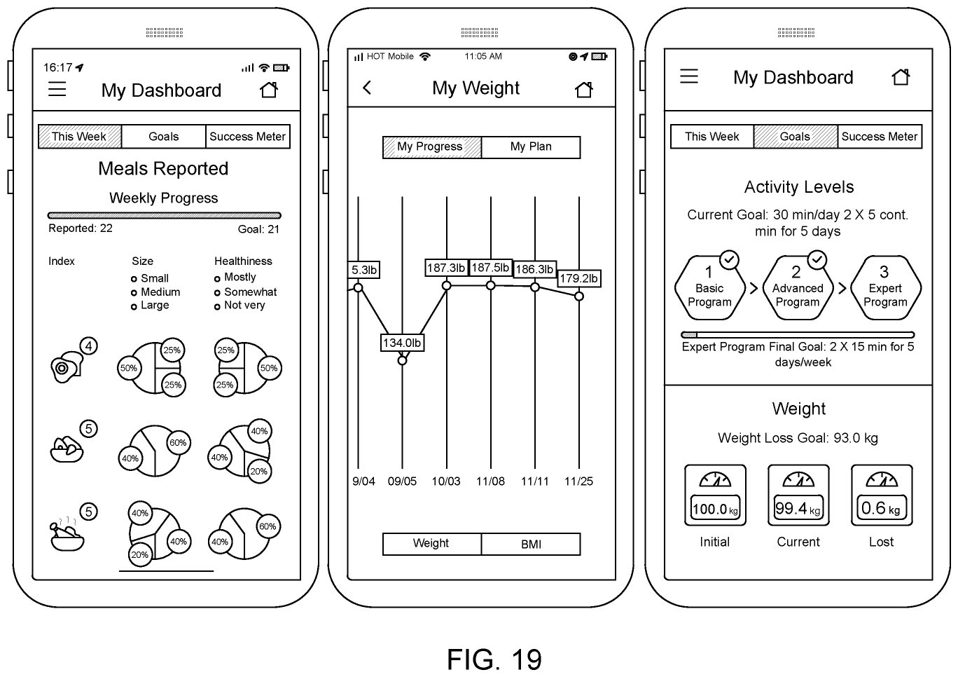

FIG. 19 illustrates an example of In-app Patient Data on display screens in accordance with an embodiment. In an embodiment, all the data collected from the patient is available within Sweetch app (and may be stored as noted with regards to systems 100, 350, as well as updated as needed). The data may be presented in detailed, aggregated and graph view to enable the patient track and be aware of his progress to the details. Personal data is secured by another layer of protection-username and password—in accordance with an embodiment. The patient can store or share this data with his HCP or anyone else.

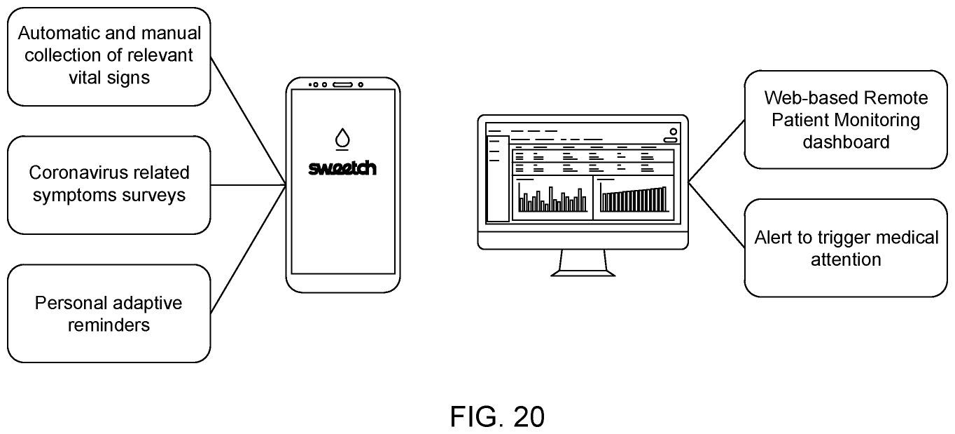

FIG. 20 illustrates an example of a Sweetch Offering for epidemics, such as COVID-19 and/or other pandemic occurrences in accordance with an embodiment. When epidemics (such as COVID-19) spread, hospitals are expecting a surge in visitors to emergency rooms, which result in a challenging additional burden to the already-overstretched medical system. In an embodiment, Sweetch's clinically proven AI-powered Remote Patient Monitoring and Disease Management Optimization Platform enables medical teams to remotely monitor, follow and improve clinical decision-making and outcomes of patients with chronic diseases in a highly personalized and scalable way. Further, there is Sweetch Value Proposition to epidemics: Sweetch helps keeping patients who are most susceptible to an epidemic the disease complications at home while still providing them with optimal care, minimizing disease spread, and infection risk. Sweetch also enables medical teams to remotely monitor, follow and improve clinical decision-making and outcomes of epidemic disease patients that also have chronic diseases in a highly personalized and scalable way.

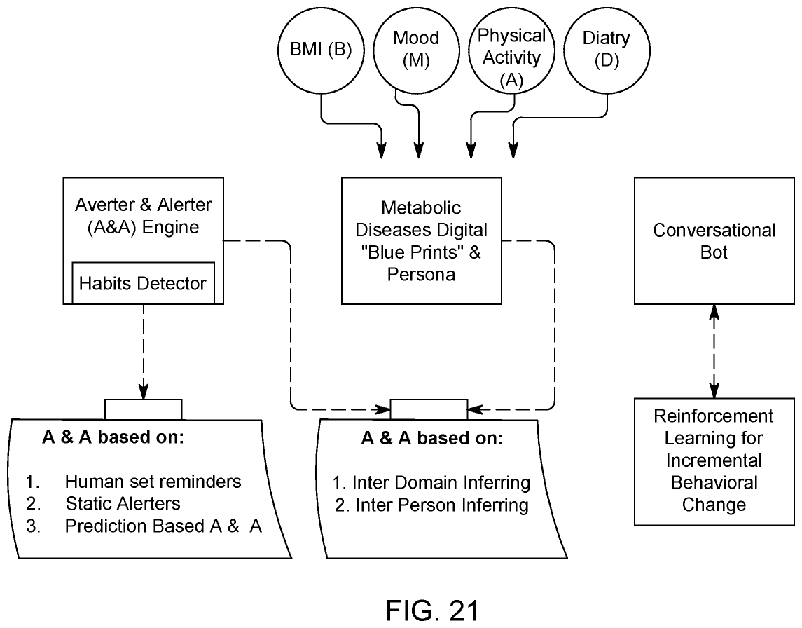

FIG. 21 illustrates an example of a Block Diagram of modules (described herethroughout) involved in innovation points discussed in accordance with an embodiment herein. As shown, modules may include Alerter & Averter AI module, and Diseases module, and a Conversational Bot.

FIG. 22 illustrates an example of Sweetch prediction flow in accordance with an embodiment. As described in greater detail throughout this disclosure, the prediction flow may include data collection, data analysis, and predictions.

FIG. 23 illustrates an example of Sweetch intervention flow in accordance with an embodiment, which includes sensing and collecting data, analyzing data, and responding.

FIG. 24 illustrates an example of Sweetch logic tier architecture 260 in accordance with an embodiment. As shown, the architecture 260 includes an interaction DSS module/system 262, an interaction hub 264, and a Behavioral Analytics AI (BA-AI) system 266.

In accordance with an embodiment, the Behavioral Analytics AI System (BA-AI) system 266 may be composed from one or more of the following modules: Daily Messages Reinforcement Learning AI, Habit Detector AI, Alerter & Averter AI, and Multiple Goals AI Reinforcement Learning for Incremental Behavioral Change.

### Daily Messages Reinforcement Learning AI

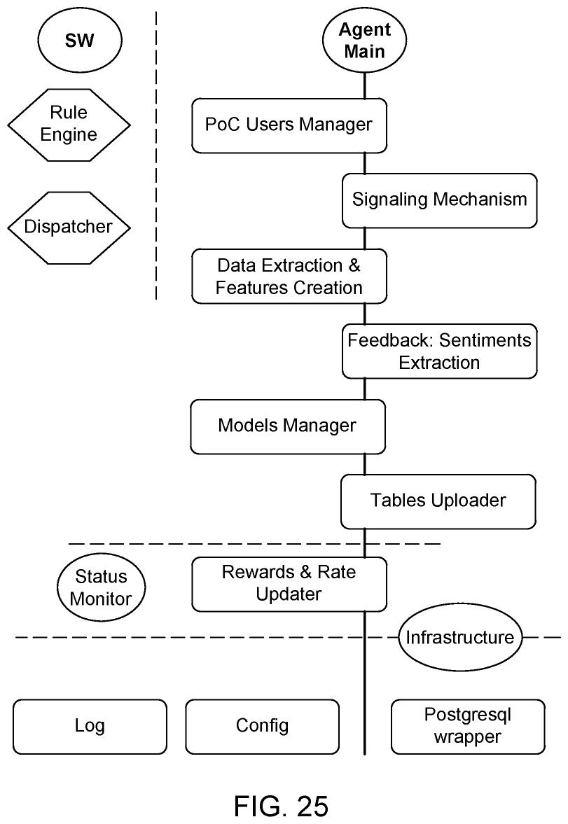

Daily Messages Reinforcement Learning (RL) AI is an algorithm that matches daily messages to the patient. For each and every patient the RL AI learn what are the messages that the patient responds to the best. Patient behavior is collected, normalized and then analyzed by identifying specific indicators as well as patterns/trends in patient performance data. Indicators such as response time, actual physical activity time, physical activity time of day, changes in weight, changes in schedule, explicit data input by patient, features form the user's blueprint and more are monitored and analyzed in order to select the best model which will push the patient to complete its/her planned physical activity. The models incorporate a feedback mechanism that over time, prefer messages that cause the user to perform the best. Since the AI reacts in real life, there is always need to combine exploration mode which explore which are the best models together with exploitation mode that exploit the learned motivators by sending the preferred messages for the user, hence, pushing him to perform more physical activity (see, e.g., FIG. 18, as well as FIG. 19, which show examples of Daily Messages sent to the user and the amount of physical activity perform in reaction to the sent messages over days and hours.) The icons represent different sentiment tone of the sent messages while the physical activity is color coded, e.g., one color represents less physical activity while another color represents more physical activity performed, and yet another color represents no physical activity at all. FIG. 25 illustrates an example of Daily Messages RL AI in accordance with an embodiment. The Daily Messages RL AI method can be applied for selecting/constructing any part of the message: timing, content, sentiment, components of the message (e.g., weather, calendar, emojis, etc.). In accordance with an embodiment, Daily Messages Reinforcement Learning AI includes:
RL—reinforcement learning system. Training is ongoing as more experiences (i.e. interactions with the user) are collected
Evaluates of the score on each arm/action which are used for selecting/constructing the messages may be performed by any of the RL algorithms. For example Least square fit (LSF)—so slope dictate prediction direction, Q-learning methods (e.g. DQN), contextual bandits algorithms (e.g. LinUCB).
Day to day models evaluation
Fierce competition, evaluation & evolution
Reports & Tools that count winners of models & Sentiments
Exploration: Give Model/Sentiment/Message/Content/Timing a Chance

Decay Process (i.e. recovery mechanism)—for un responded sentiments/content/timing/components-per user. The recovery mechanism may be based both on evaluations of the user responses to the specific arm or action and on how recently the arm was previously used. The recovery mechanism can be based on Gaussian process kernels, parametric approaches (e.g. exponential decay) or simple FIFO mechanisms

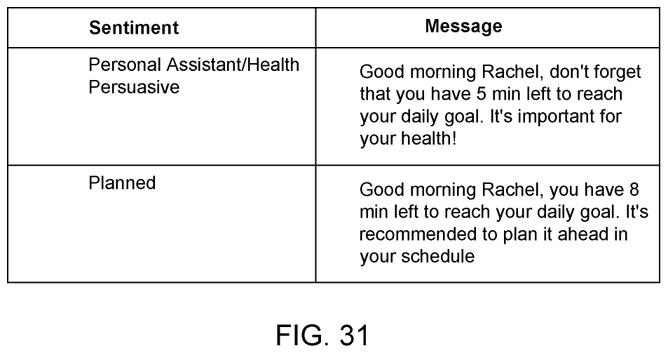

Examples of sentiments and messages that may be delivered/sent to a user are illustrated in FIG. 31. As shown in FIG. 31, for example, sentiments may be direct towards a personal assistant/health persuasive concepts, or planned concept.

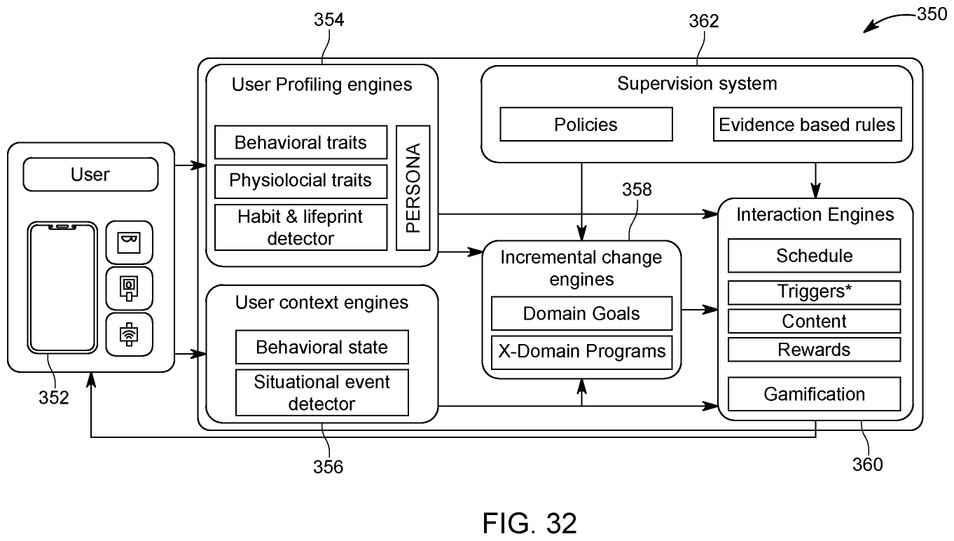

FIG. 32 illustrates an exemplary embodiment of a system 350, in accordance with this disclosure, which enables the herein described prediction, prevention and management of chronic and autoimmune diseases. As shown, the input is collected from the user via the mobile device, the application provided thereon, and/or any/all connected devices at 352. The data is processed by one or more user profiling engines 354 and one or more user context engines 356, which includes a behavioral state estimator and a situational event detector, to generate a feature or user state vector which is used for down the line processing by the system 350 to determine, e.g., user goal(s) for each domain and the Incremental Change Programs (ICPs) of the incremental change engine(s) 358 to guide the user toward those goals. Based on the one or more user profiling engines, a digital blueprint of the user is generated. The ICPs might be intra domain and cross domain, for example, in accordance with an embodiment. The system 350 generates for the user a multitude of ICPs. For example, the user might have an ICP program for physical activity, for weight management and for improving meals habits. The ICPs are generated based at least in part on data processed from the one or more user context engines, the digital blueprint of the user, and a predefined set of goals. The user's state vector (digital blueprint of the user) from user profiling engines 354 and the user context from user context engines 356 together with predefined set of one or more user goals and ICPs from incremental change engines 358 are used as an input to the Interaction Engine (IE) 360. The IE 360 is designed to determine a specific interaction to be delivered to the user. In embodiments, both the incremental change engines 358 and the IE 360 may receive input from a supervision system 362 which is designed to enforce pre-defined policies and implement evidence-based intervention program(s). As described in greater detail below, the IE 360 may include a scheduler which determines the optimal times of intervention for each domain and the next domain for the interaction. Once the time and domain of interaction are determined the IE 360 assembles the specific message that will be sent to the user.

Further details regarding each of the engines and features of the system 350, according to one or more embodiments herein, are described below:

### User Profiling Engines 354

The system continuously generates new insights, features and characteristics on a patient, enabling a deep understanding of the patient's behavior and understanding his behavior implications. This includes generalization of the collected data (habits, reactions, routines, preferences and more) that may represent, classify, and categorize each individual as a persona or as a combination of personas. In accordance with an embodiment, such may be implemented by creating digital “Blueprints”, which may include a set of unique traits and features that define the user. This unique per-user digital “Blueprint” enables the disclosed systems and methods to capture the core of each patient and then understand his/her behavior, motivations and later on, allows the platform to predict and explain his/her behavior. In addition, behavioral implication and context are defined and identified erecting a digital typical day and week “Blueprint”. Among others, this enables the disclosed platform to target negative behavior that has direct implication on the patient's health condition. This capability is based on the use of machine learning algorithms to first detect negative behavior, by understanding the context of a specific behavior in relation to the patient's health condition, and second, to efficiently encourage healthy habits by avoiding that negative behavior, all with the use of an AI Reinforcement Learning approach.

### Behavioral Traits

The data collected by the system is used to identify inherent traits of user behavior, for example “this user is physically active”.

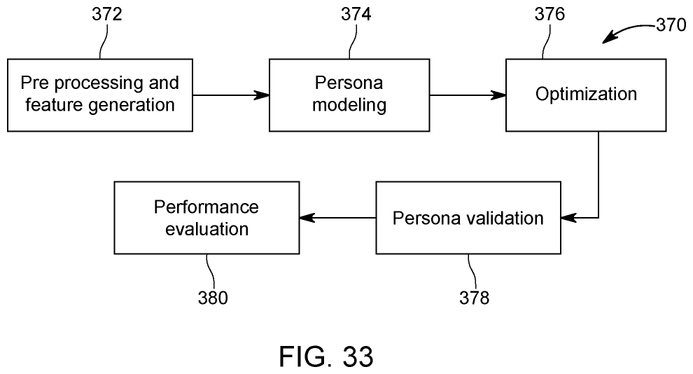

The identification of the user persona is performed by a clustering system in accordance with an embodiment. An example of a clustering system 370 is described in FIG. 33, which may include the following implemented by one or more processors or modules in accordance with an embodiment (which are further described below): at 372: Pre-processing and feature generation; at 374: Persona Modeling; at 376: Optimization of hyperparameters and feature selection; at 378: Persona validation; and at 380: Performance evaluation.

### Pre-Processing and Features Generation 372

In accordance with one embodiment herein, pre-processing and features generation is performed based on the steps described with reference to FIG. 2. FIG. 34 shows another example of pre-processing steps 382 that may be performed by the system. Data may be loaded at 384. As described with reference to FIG. 2, the steps 382 may include an aggregate step at 386, a clean step at 388, and a create features step at 390. Further steps may include impute data at 392, handling categoric features at 394, and normalizing data at 396.

### Modeling 374

Modeling is performed by applying one or several clustering algorithms on the features generated by the previous step (i.e., pre-processing and feature generation at 372), in accordance with an embodiment. The algorithms may include, but are not limited to K-means, Agglomerative, DBSCAN, Mixture models and others.

### Optimization of Hyperparameters and Feature Selection 376

As briefly described with reference to FIG. 3, data from the modeling step 374 is then optimized and features are selected at 376. Feature selection may be performed, in accordance with embodiments herein, by any of the following methods: forward selection, backward elimination, genetic algorithms. Hyperparameters optimization may be performed by grid or random search or by any method of Bayesian optimization, for example, in accordance with embodiments.

### Persona Validation 378

The data from 376 is then validated at 378. Validation of the persona is performed by comparing the personas generated by the different models and by “K-fold cross-validation”, where the personas created by same or different models on different folds of the data are compared for validation.

Thereafter, performance evaluation is performed at 380.

### Physiological Traits

Similar to behavioral traits, the disclosed system also extracts (when possible) physiological traits of the user. An example of physiological trait might be “glucose levels of this user are not significantly impacted when he performs physical activity of type walking” or “this user has a short deep, stage 3, sleep. In accordance with an embodiment, the ability to extract physiological traits depends on user having a relevant connected device, such as CGM for glucose monitoring, smart watch for sleep monitoring, etc.

### CGM Analysis

CGM devices measure the glucose levels of a user all the time or at regular time intervals throughout a day, e.g., usually sampled at 5 minutes intervals. The measured CGM signal may be decomposed into components including the baseline CGM, the dawn effect, responses to daily activities such as physical exercise, meals etc.

A Discrete Wavelet Transform (DWT) is used to identify different components of the signal, in accordance with an embodiment. The specific mother function for the DWT can be configured and the signal details at different levels are extracted by the DWT. These details at different levels, when combined, construct the full CGM signal (together with the last level approximation). The system combines the signals from different details levels of the DWT transform into relevant glucose responses of the user. For example, details level 4 and 5 from the DWT transform may be combined together and constitute the glucose response of the user to meals, while the last level approximation from the DWT is constitutes the baseline glucose response of the user. From each component, a multitude of features can be extracted, representing important parameters of the glucose responses of the user. For example, the features set comprises, time to peak glucose level, the peak value, time to baseline glucose level. These features are extracted from each component separately (i.e. from the response to meals and from the response to physical activity, etc.) In accordance with an embodiment, the features may be directly used for downstream processing or may be used to identify inherent physiological traits by methods similar to those described with reference to the behavioral traits section herein. Downstream processing, may include statistics, correlations or predictions of user glucose responses.

### Habits and Lifeprint Detector/Habit Detector AI

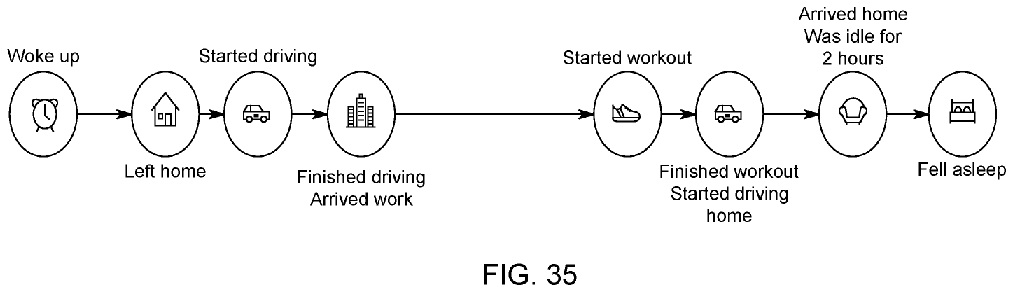

The system identifies the user's lifeprint and a typical day and a week schedule, in accordance with embodiments herein. Such identifications may include a user's important and/or frequently attended places or locations (e.g., home and work location, favorite coffee shop and gym). Further details regarding collecting such data, habits, etc. are described below with regards to the A&A AI unit. In accordance with embodiments, the system may also identify a user's sleep and commute patterns, weighing patterns, etc. as shown in FIG. 35, for example.

### Lifeprint

In an embodiment, the set of important/frequency places may be identified by collecting location information of the user for a predefined period of time and then analyzing it to deduce the locations most frequently visited by the user (important places). This may be achieved by applying different methods, e.g. KDE, meanshift The identified important/frequent places are labeled by using standard APIs (e.g., Google places), for example, according to embodiments herein. The system then may combine the identified imported places with other location in the area of the user and incorporate those in the interaction and messages for the user as can be seen in FIG. 8.

### Day and Week Schedule

The day and week schedule are extracted by combining the location information with movement sensors information gathered from the mobile device and information from connected devices, in accordance with embodiments. The identified information comprises, but is not limited to:
Going to sleep time
Wakeup time
Go out time
Commute times
Weighing times
Meals times
Physical activity times
User Context Engines
Behavioral state

Behavioral state of the user contains information about the user's recent patterns of behavior which might be coherent with his Behavioral profiling and might be different. For example, a user might be a heavy commuter, but he did not go out of his house this week. Users context might also include features describing his previous activities within the Sweetch app (e.g. weight reporting, meals reporting, etc.) and responses to previous interaction from the system.

### Situational Event Detector

Event detector engine is characterizing user's current situation. For example, the user is currently at supermarket, or he is currently driving, sleeping, walking, etc. . . .

### Incremental Change Engines 358

The incremental change engines 358 may be utilized to set goals, in accordance with embodiments herein.

The final goals for the user may be set by the user himself either during an onboarding process or by answering a questioner in the app. Goals may also be set by the Supervision System 362 as described herein. In accordance with an embodiment, once a goal is reached, or, on the contrary, the user is unable to reach his goal on a certain domain, the goal may be changed by the system to a different goal. In accordance with embodiments herein, the decision to change the goal as well as a new/different goal may be set automatically by the system. For example, the next goal may be based on the time which it took the user to reach his previous goal.

### Incremental Change Programs (X-Domain Programs) of the Incremental Change Engines 358

The goal of the ICP is to guide the user through a series of intermediate (daily and weekly) goals on his way to a pre-defined final goal. According to embodiments herein, intermediate goals may be daily, weekly, and/or without a predefined time increment. The goals may be scalar (e.g. increase daily physical activity by 5 minutes) or vector goals (e.g., increase meals reporting and increase meals healthiness). The ICP with their intermediate goals may target a single domain or multiple domains (e.g., increase physical activity by 30 minutes and reduce weight by 1 kg next week). In accordance with an embodiment, there are two mechanisms for selecting or constructing the ICP. In both cases, the system uses the features extracted by the user profiling engines 354 and user context engines 356 to select or construct the personal ICP. The ICP may be selected by the system from a set of predefined programs based on the user profiling results and his context information and then updated (i.e. reselected) once the user context has been changed, in accordance with an embodiment. In another embodiment, another mechanism for the ICP is constructing the program on-line, constantly adjusting the next incremental step based on the updated user profiling and context. This may be achieved by using Reinforcement Learning (RL) algorithms for selecting the next increment.

### Interaction Engines 360

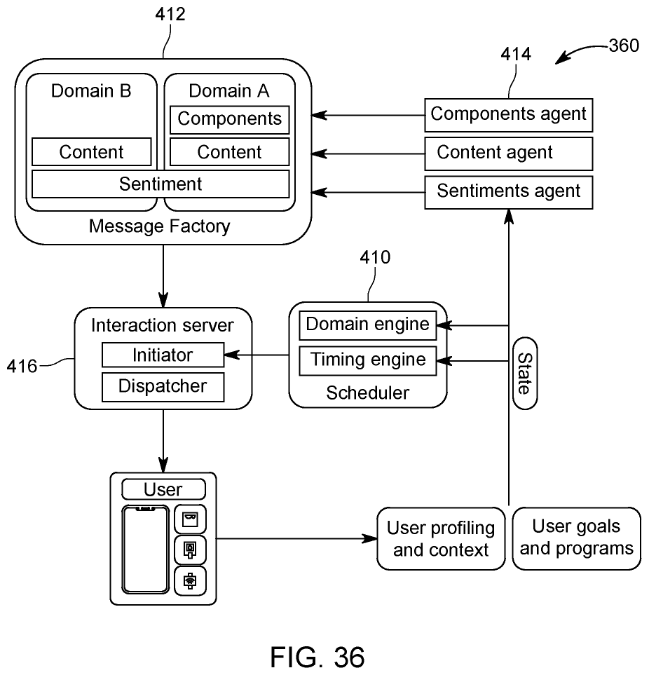

The interactions engines architecture is shown in FIG. 36. In accordance with an embodiment, user profiling and context information is combined with the output of the incremental change engines 358, namely the user goals and his ICPs for in each domain into a user state. In an embodiment, the scheduler optimizes the time and the domain of interaction and the specific interaction is then constructed. The system is designed to perform several types of interactions with the user. Such system interactions may comprise, but are not limited to: (1) content recommendations, (2) triggers, which include recommendations, reminders, alerts, averters, and (3) rewards, which include encouragements, badges and achievements. The message factory is the part of the system where the message for a specific interaction is constructed. Interaction may be a push notification, message shown in a pre-defined areas in the app, and/or as a response to user's activity within the app. An interaction may be carried out also outside the app, for example by sending an e-mail to a user.

### Scheduler 410

Scheduler 410 is a set of engines selecting the domain and the time of interaction. Domain engine quantifies the probability of user interaction in each domain. The ranks are used to determine the domain in which the user should and should not be interacted. The optimal time for interaction may be selected by the system for each user and in each domain based on the user state. The optimal interaction time might be selected by RL algorithm, where (relevant parts of) user state is used as the state for the RL algorithm and the different hours for the next interaction are the actions to be selected by the RL agent. To optimize response to user situation and provide real time interactions, the system, in a different embodiment, may have a binary action of performing or not performing an interaction with the user at the current moment. In cases, when the decision is based on the RL agent, it will have a binary action. Both the domain and timing of the interaction are controlled and might be overruled by the Supervision System later on in the Initiator.

### Message Factory 412

Message factory 412 constructs messages by filling in fields in pre-defined templates. Message construction is controlled by at least three engines 414: a content agent engine, which selects the template, a components agent engine, which selects which components will be added to the interaction, and a sentiments agent engine, which controls the sentiment or tone of voice to be used in the specific interaction. For each engine, the selection is implemented by training a different RL agent to select the optimal action, in accordance with an embodiment.

### Interaction Server 416

Interaction server 416 oversees and manages all the interactions with the user. It contains the Initiator, which combines the information from all the interaction engines and from the Supervision system to decide on the specific interaction to be performed. The time of the interaction may be based, in accordance with one or more embodiment, on the optimal time determined by the Timing engine, or it might be event based (e.g. user is active in a specific part of the app) and/or time based (e.g. pre defined time for medication reminder). The Dispatcher is the part of the system performing the actual interactions with the user and is the gateway for all the communications with the user.

### Supervision System 362

The supervision system is in charge of enforcing pre-defined policies and implementing evidence-based intervention programs.

### Policies

There are multiple types of policies which might be enforced for a specific user, in accordance with embodiments herein. For example, a system may enforce maximal cap on the number of notification messages sent to a user to prevent alert fatigue. A different policy may be enforcing that the medication reminders are always sent, even if system prefers to schedule a different interaction with the user.

### Evidence Based Rules

Evidence based rules are programs, plans, limitations, goals or any other type of guidance which is derived from standard guidelines for supporting patients. According to embodiments herein, these evidence based rules may comprise but are not limited to healthy meals habits, weight loss goals and physical activity plans. For example, CDC Diabetes Prevention Program (DPP) contains (among others) guidelines for amounts, duration and intensity of physical activity.

### Monitoring and Management System

### Management System

For care providers and healthcare organizations, Sweetch offers a web-based management dashboards that enables both population and individual views as described in previously with regards to FIG. 16 and the Remote Patient Monitoring (RPM) and Management Dashboard.

### Monitoring System

In accordance with embodiments herein, the system includes an interface which allows a dashboard type of display, dividing users into a predefined set of groups (example: those at home, those without physical activity those of specific persona type). The interface is designed to show detailed information regarding the patient (e.g., list of locations, activities, reports), giving the operator all the necessary data he needs in order to be able to monitor the performance of the system. Data from the user is received and processed by the server, categorizing each user into a predefined group, based on their received data.

In embodiments, the operator, entering the interface, is able to monitor the system performance as a whole, based on different segments (filtering the data according to different system parameters) and also on a user by user basis, based on their status and previous notification history.

The system allows setting policies for the whole system, per specific group of users (either predefined or a group identified by the system) and in particular on a per user basis, in accordance with embodiments herein.

### Additional System Components

The system may include additional components, including, but not limited to: educational modules, gamification modules, and Social Support modules, as previously described in this disclosure.

### Implementation Description

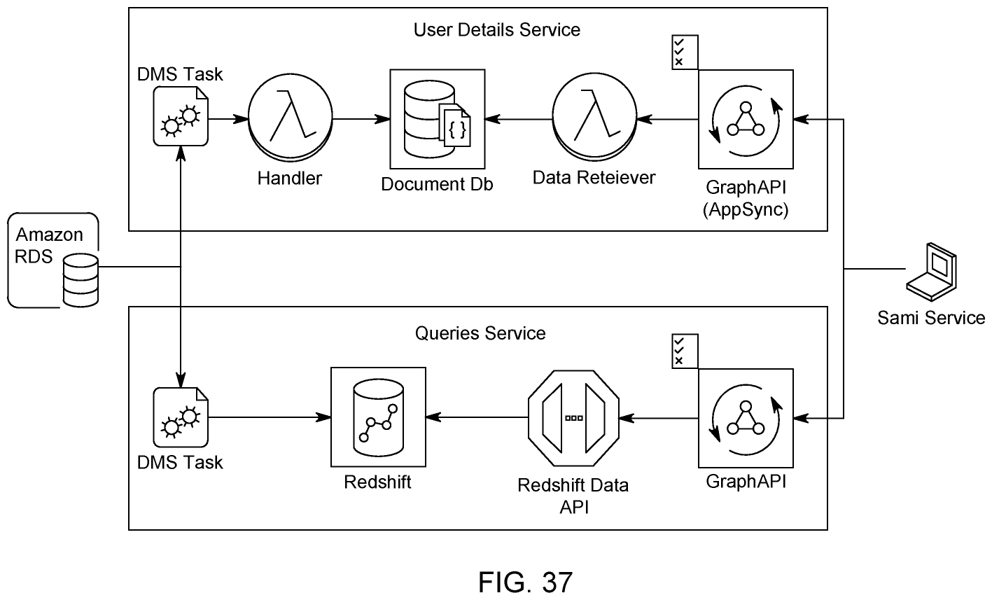

FIG. 37 shows an implementation of a generic logic of the system, in accordance with an embodiment. The upper part of the flow corresponds to user state extraction as described for example in user profiling and user context sections. The lower part of the figure shows a use case implementation. An example of a use case might be the Scheduler selecting the best time and domain of interaction, message constructed by the message factory is initiated and dispatched by the Interaction server. In order to have maximal flexibility when implementing the logics described above we have defined SAIL-Sweetch Artificial Intelligence Language. It gives full flexibility of splitting the implementation of any logic between the server side and the client side.

### SAIL

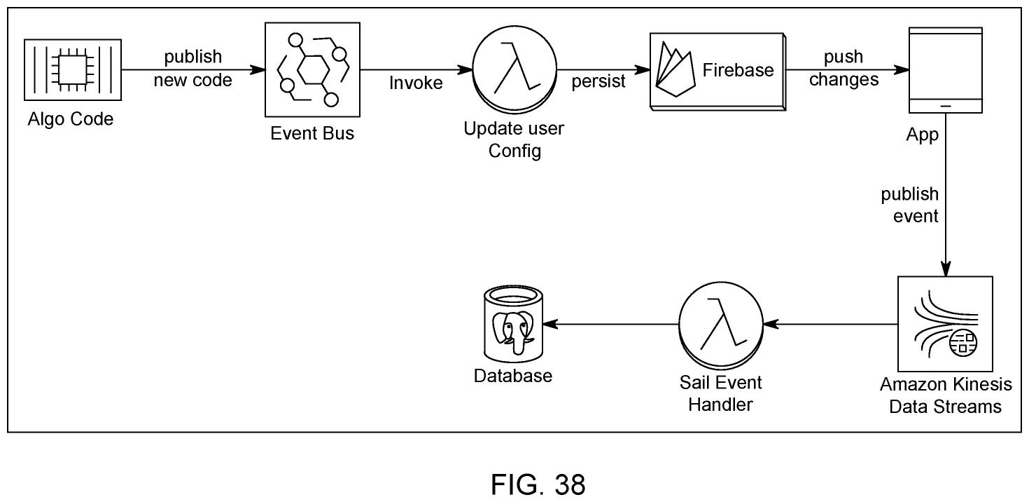

SAIL allows injecting fully personalized logics to each user. The flow of the SAIL implementation is shown in FIG. 38. When the personalized logic executed on the client needs to be updated, i.e., the system marked in FIG. 38 by “Algo code” decides to “publish new code” for a user. An example of such a trigger might be a change in the user behavioral traits (i.e., change in user's persona), or update to timing engine or any other change in the logic executed on the client. This decision is transmitted via an Event bus to a logic executing user config update. The logic updates the relevant tables for the specific user in the firebase. The update may contain any logic to be executed on the client.

Once the client becomes aware of the awaiting logic update, it publishes the event to notify the server that the new logic was received and updated. This mechanism closes the loop of synchronizing the server and the client state of logic. In accordance with an embodiment, all the client decision (made by the logic) are transmitted to the server side through Amazon Kinesis Data Streams service and are recorded to the database.

AI Based Alerters and Averters that Drive Behavioral Change

The platform analyzes and predicts negative behavioral and habits that lead to chronic disease deterioration, by providing warning, and/or corrective actions for each patient according to the pre-identified negative behaviors. In accordance with an embodiment, the disclosed system and method includes a Bad Habits Detector which is part of the Alerter & Averter AI (A&A AI) unit, for automatically detecting recurring habits or patterns of future behaviors. The list of these events may be generated by a supervised list with a predicted risk score per event, according to one embodiment. The A&A AI unit is capable of providing a-priori alerts by recommending alternative actions to take. Here are some example use cases where the A&A AI may detect and intervene:
An elderly patient who has a health habit of eating Humus at 12:20 pm every day—The AI will identify this bad habit, and recommend and encourage him to go to the nearby salad bar at 12:00 instead.
For several consecutive days, a patient worked late hours and went to sleep late, after eating late dinner, hence, skipping his regular exercise—As a result, the AI will notice the problematic trend and will notify the patient to pay attention and go back to his healthy daily routine.

According to embodiments herein, the technology includes: a Set of supervised habits, a Habits Detector, ETL process for Habits data streaming & Features extraction in real time, Alerter & Averter (A & A) AI engine, and collaboration with the Persona unit. According to one embodiment, the set of supervised Habits may be selected from the following domains: Disease management, Dietary Intake, Medicine Intake, Mood & Energy Feeling and Physical Activity. According to an embodiment, the Habits Detector may include: NLP prediction algorithms together with Genetic optimization methods; clustering algorithms such as Dbscan, HGMM, Hierarchical K-Means/PCA, Birch; a series of similarity/dissimilarity algorithms such as Dynamic Time Wrapping and others; implementing Hybrid recommendations systems such as factorial Machines; and identifying regular good and bad Habits as well as bad outliers using methods as described herein in order to perform prediction or wisdom inferring.

In an embodiment, the Alerter & Averter (A & A) AI engine is configured to process the Habits Detector outcomes to a meaningful messages/Bot discussion to be sent to the user Just in Time. In accordance with an embodiment, the A&A output may be one or more of the following types: Static/Dynamic, Prediction based, and Manually/Human set. Together with the Persona unit, the Alerter & Averter (A & A) AI engine may be used in order to send one or more of the following: Inter Domain Inferring, Inter Person Inferring, and Tools and Dashboard for Inspection of the A&A statuses as well as the user's reactions & responses over time.

### Chronic Diseases Digital Persona and Characteristics

Healthy life cycle includes various elements: Mental healthiness, physical wellness as well as vigor active mind and psyche for example. A balanced life style requires monitoring and feedback of all such domains.

Generation and utilization of internal markers that genuinely represent the individual motivations and drivers as a persona. By achieving better characterization and classification, Sweetch provides more personalized health recommendations to patients' conditions. Optimal characterization of user's digital persona is imperative for being able to help users. Insights of each patient include, as much as possible, different aspects of the user's daily life, habits and routines, and internal and external traits.

The basic of this understanding is achieved by creating the aforementioned digital “BluePrints”. As a result, Sweetch is able to guide the user toward a sustainable behavioral change. It enables to predict the user challenges, provide him/her hyper-personalized relevant recommendations, and build a long-lasting behavioral change that will improve the patient lifestyle and health.

Several aspects of the healthy life cycle, which are referenced as BMAD, are considered:
B—BMI/Weight space
M—Mood: psychological and general feeling and wellness space
A—Activity: physical activity space
D—Dietary: meals reporting and dietary habits space

### The Technology Includes:

Detection and extraction of meaningful features that are Behavioral Change (BC) and Mood (M) related.
Detection and extract of meaningful features that are BMI/Weight (B) related/Meals (D) related/Activity (A) related
Detection and extract of meaningful features that are Leaderboards, Competitions and Challenges related.
Algorithm that characterize single descriptive number per domain.
Clustering per domain using several methods such as: Dbscan, HGMM, Hierarchical K-Means/PCA, Birch.
Multi Domain clustering and Persona consolidation using several Clustering Methods as well as Soft Clustering or Weighted Dynamic Mean-shift.
Clusters Optimization using genetic algorithms search methods.
Personal “Blue Print” and Statistical Perspective dashboards.

The disclosed systems and methods may also provide multiple Goals/Rewards AI Reinforcement Learning for Incremental Behavioral Change, in accordance with embodiments herein. Overcoming bad/cumbersome habits is hard. One can acquire good habits and adhere to it, if she/he can follow and acquire small achievable victories. These accumulations of many small successes will change the user perceived self-efficacy drive a cognitive development process eventually resulted in a sustainable change. In order to motivate and drive the user, several goals which might be achievable and actionable by an incremental step by step process may be determined and implemented. To achieve such goals, the disclosed sophisticated AI engine is based on Reinforcement Learning with multiple targets mechanism. The AI engine empowers the patient/user by dynamically adjust to his/her needs, define appropriate goals and provide guidance for his/her personal journey to achieve these goals. The engine will continuously revisit the goals set and may change them based on real-life data, directing the user to have a course of wins that will lead to sustainable behavioral change. This is an open problem in the field of algorithmic reinforcement learning, and thus this disclosure aims to address such challenges. The AI engine converses through the Bot. The technique of multiple goals is implemented to various domains.

When dealing with dietary/meals habits, one can think of various possible goals. Some of the goals are easier to achieve than others, some are achievable in the long run while others are easier for certain people as a short-term goal; for example: Start reporting any food consumption/Eat smaller portions/Eat dinner in earlier time and more. In the field of pharmaceuticals, goals may be dictated by the specific medication instructions or by the HCP—warnings, possible side effects as well as the fear for adverse events. All as stated in the pharmaceutical instructions and or the HCP's; for example: Start taking the medication/Report Any adverse effect when occur/Report general feeling/mood and influence at the intake point.

The technology includes, according to embodiments herein:
Goals/Rewards setting in the various domains: e.g., disease management or meals and nutrition
ETL process for data streaming and Features extraction in RT
Predictors Algorithms
Net alternations; and
Tools & Dashboard for Inspection of the user's progress & improvements.

In an embodiment, the predictors algorithms may include one or more of the following: Deep Neural Network: Encoder, Decoder, Attention mechanism, Self-organizing Map (SOM) with/or Supervised Attractors; Boosting mechanism with Logistic extreme gradient boosting; and Hybrid recommendation systems using Factorial Machines. In an embodiment, Predictors Algorithms may include one ore more of the following: Curiosity Driven Reward, Attention component, and Greedy Max Policy.

### The Interaction Hub 264

The Interaction Hub is responsible for all the communication with the users through all the communication channels. These communications provide the user with optimal guidance and motivation on how to better adhere to the intervention program while offering them choice which best suits their life habits, past behavior and current context (e.g. at work, weekend, roaming etc.). The Interaction Hub is designed to utilize personal behavioral and contextual data in order to personalize best practice disease management protocols which are consumed as “recommendations”, “plans” and “notifications” which motivate the patient to improve their condition, in accordance with embodiments herein. According to embodiments herein, the interaction hub collects data from all data streams (from all sources) and provides feedback to the user (including guidelines, alerts, reminders, conversation etc.). All communications to/from the users as well the various Sweetch modules are passing through this module.

In accordance with an embodiment, specific sub units may include:
Patient Reported Outcome Measures (PROMs);
Notification Dispatcher, which sends different notifications to the user;
Bot communications, which enables conversation service; and
Device connectivity, which enables easier reporting and measurements seamless streaming. See also Devices Connectivity below.

One of the modules within the Interaction Hub may be a conversational personal assistant Bot, such as shown in FIG. 21. In accordance with an embodiment, this is a script-based Bot that enables the user to perform free natural conversation as well as reporting. The backend may include a set of NLP parsers in order to segment the perceived conversation and stores it Sweetch Power Data unit.

In an embodiment, the technology includes:
Implementation in several domains for reporting: Dietary Intake, Medicine Intake, Mood and Energy Feeling;
Minimal clicks on selected domain;
Open reporting based on selected domain;
ETL process on selected domain;
NLP segmentation algorithms that detect useful information from selected domain;
Tools & Dashboard for Inspection of the user's progress & improvements; and
Reinforcement Learning AI from the Multiple Targets/Rewards AI Reinforcement Learning System for Incremental Behavioral Change assignment.

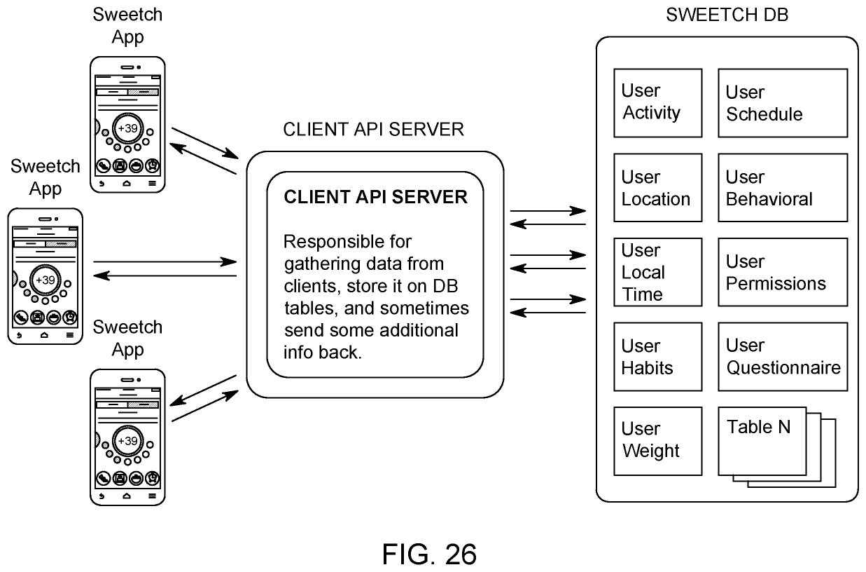

FIG. 26 illustrates an example of a Client API Server in accordance with an embodiment. According to embodiments herein, the Sweetch API Server is mainly responsible for data gathering from the clients (apps), back into the Sweetch backend. In an embodiment, the architecture is known as client-server model—where it is always the client who can initiate a request to the server and never the opposite. In an embodiment, the server publishes a wide variety of API calls implemented using HTTPS protocol that the clients use for sending data to the server, and sometimes collect info or guidance from the server. According to embodiments herein, for using any of the calls, the clients must first be authenticated through an OAuth2 compliant authentication request. Once the server successfully authenticates and validates the incoming request, it sends a special access token in response, for example. In an embodiment, from the moment the client is authenticated, it uses this token for every single request to identify itself, and the entire communication between the client and server is encrypted by SSL. In one embodiment, various requests coming from the clients populate multiple DB tables with updated information, that is later processed and analyzed by other offline services like BI server, behavioral analytics engine, locations server and more.

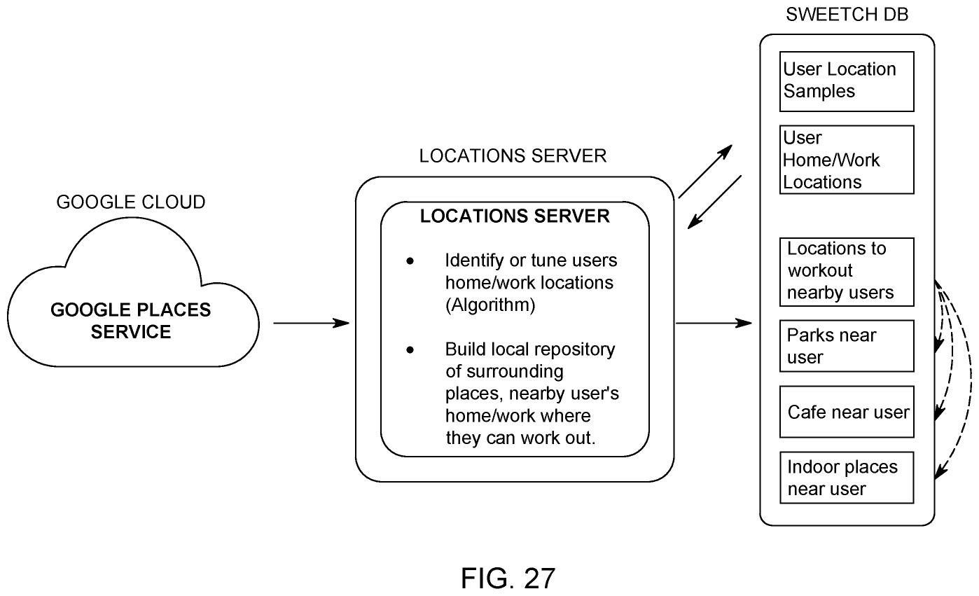

FIG. 27 illustrates an example of a Location Server in accordance with an embodiment. According to embodiments herein, the Locations Server has two primary responsibilities: identify user important places and compile a list of recommended places to be used as part of the system interactions in the proximity of important places. Important places comprise home, work, gym, favorite coffee shop and any other type of location a user frequently visits. In accordance with an embodiment, this task frequently runs an algorithm to identify or fine-tune the user's important places locations and refines the recommended places around the “anchor” important places, such as home and work. These two “anchors”, are used when Sweetch sends accurate suggestions for places where the user can perform activity (e.g. a park next to his house, walking to a specific coffee shop near his work to meet the user daily goal), according to embodiments herein. Important place can also serve as recommended place. For example, when user is home, the system might recommend him walking to his favorite coffee shop (which is both important place and recommended place related to the home anchor). The accuracy of such messages has been proven for being vital for increasing the users adherence to their goals.

According to embodiments herein, the Location Server is configured to gather surrounding locations. In an embodiment, this task is in charge for gathering surrounding places around the user home and work locations (or other important place, where user spend a significant amount of his time), where the user is able to perform activity. In an embodiment, for this task, the server connects to a known service, such as “Google Places”, and applying set of queries builds a database ordered by categories of such places. All data gathered is then available for other components of the Sweetch solution, like Interaction DSS 262, behavioral analytics engine, BI Server and more.

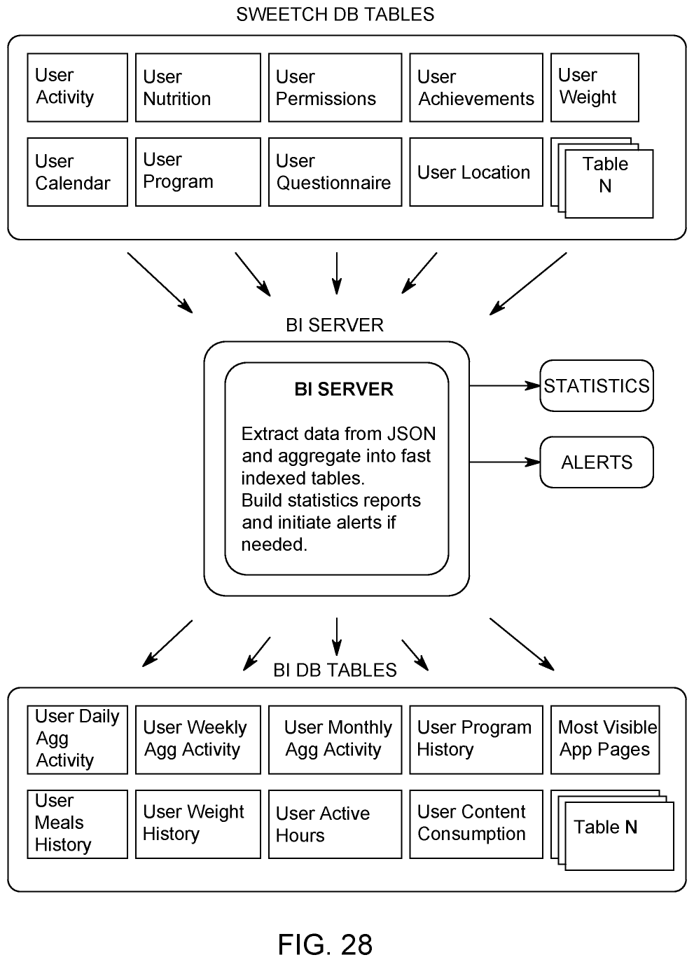

FIG. 28 illustrates an example of a BI Server (Behavioral Intelligence Server or Engine) in accordance with an embodiment. According to embodiments herein, the BI server is responsible for 3 major tasks: Data Parsing and Aggregation, Data statistics (Business logic), and Data Alerts (Business logic). With regards to Data Parsing and Aggregation-Since all data coming from clients (i.e. raw data) arrive in JSON format, it is the BI Server responsibility to extract mass data into fast-indexed tables for deeper usage. In embodiments herein, the BI Server works offline 24/7 and extract data into separate columns within well indexed tables. The BI Server outcome tables may be used by several components—behavioral analytics engine, Interaction DSS 262 and admin tool (monitoring system), for example, according to embodiments herein. With regards to Data statistics (Business logic)—The BI server is reasonable to gather data for a pre-defined statistics report. According to embodiments herein, the reports are pushed to the Admins via emails or accessible under the relevant permission in any of the dashboards serving the solution. The reports frequency is adjustable in accordance with embodiments. With regards to Data Alerts (Business logic)—The BI Server is configured, according to embodiments herein, to detect values anomaly during the parsing (or aggregation of any kind), and report it. The alerts may point to a problem within the system or emphasize an unexpected human behavior.

### Sweetch Data Layer (Power Data):

Sweetch Data layer is where all the Sweetch solution data resides. There are a few DB servers, each represents and serves different type of activity. The DB's are stored on few AWS RDS entities, all part of a secured private cloud, protected by FW on a cloud server (e.g., the Amazon cloud). All DB's are configured to encrypt data at rest, using keys that are managed through AWS Key Management System (KMS).

As being stored on AWS, VPC Sweetch enjoy the benefits of some advanced security features like the ability to define security groups for any network access to the servers, or define strict policies for different roles and workforce.

In accordance with embodiments herein, the Data layer is fully operational and available 24/7. For disaster recovery, Sweetch maintains a near real-time hot replicas of the operational servers. The hot replicas are designed to immediately take over the operation when something goes wrong with the master DB.

The Data can be viewed in multiple tiers as the following:
Raw Input data from various domains
Domain specific (processed) traits—for example B related features such as weights variations.
Persona associations-composition of related traits that can describe typical prototype
Patient “Blue Print”-composition of different traits, raw data as well as different Persona. This “Blue print” resemble the inner manifestation of the patient.
Day “Blue Print”-composition of different traits and raw data, along time line that resemble the inner manifestation of the patient behavior along time line.
All input and output to/from Sweetch Power Data is performed through the Interaction Hub.

### Devices Connectivity

According to embodiments herein, Sweetch includes a component that enables the app to connect to any connected device that have a direct API. Those, among other includes, BGM, scales, heart rate monitor, Thermometer, etc., as previously noted. The usage of the connected device may be defined by the patient or by the HCP, and Sweetch make sure to remind that patient to use those, based on the treatment protocol, daily routine, and availability.

### Cloud, Servers and External API

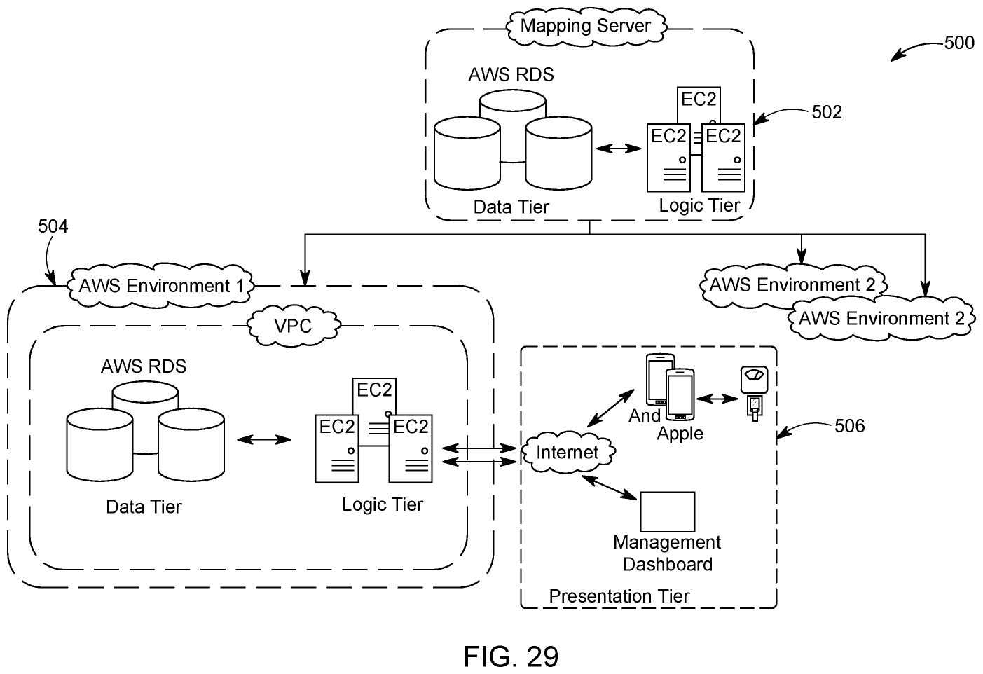

Sweetch's architecture is based on AWS environments. FIG. 29 illustrates an example of a Sweetch AWS architecture 500 in accordance with an embodiment. As shown, in accordance with an embodiment, the architecture 500 may include a mapping server 502, one or more AWS environments (e.g., AWS Environment 1 504, AWS Environment 2, etc.), and a presentation tier 506. Mapping server 502 communicates with each of the AWS environments. Mapping server 502 may include a data tier and a logic tier, for example. Similarly, AWS Environment 1 504 may include a data tier and a logic tier within a VPC, for example. In an embodiment, the logic tier is configured to communicate with a management dashboard and the user/mobile devices of the presentation tier 506 via the Internet. The logic tier includes the following services: Client API Server, Intervention Service, Interaction Service, Behavioral Analytics Engine, Location Server, BI Server, in accordance with embodiments.

### Client API Server

### Intervention Service (as Previously Described Throughout)

Interaction Service—The Interaction service is responsible for all the communication with the users. These communications provide the user with optimal guidance and motivation on how to better adhere to the intervention program while offering them choice which best suits their lifestyle, past behavior and current context (e.g. at work, weekend, roaming etc.).

Behavioral Analytics Engine-Connecting the predictability capabilities with the business rules and different models of behavior. Performs calculation and ongoing prediction of the most effective ways to motivate the user. Monitoring of the users' compliance Provides insights into methods of behavioral changes for the users

### Location Server (as Previously Described)

### BI Server (as Previously Described)

### Privacy and Security

Sweetch was developed in accordance with HIPAA (Health Insurance Portability and Accountability Act of 1996) standards in the United States legislation that provides data privacy and security provisions for safeguarding medical information. All integrations and interoperability to Sweetch are done using HIPAA standards. Sweetch is also Australian Privacy Act compliant. Best practices are deployed including, among others, AWS KMS SDK to encrypt sensitive data; SSL and https protocols; AES 256 CIPHER cryptographic protocols; full server separation between demographic data and all other data types. In addition, Sweetch is GDPR compliant and adheres to the GDPR guidelines of privacy and security, including but not limited to:

Sweetch uses few repositories for records retaining: 1. Multiple DBs on AWS under the HIPAA regulations of data security (all encrypted); 2. Bitbucket Git service to retain all organisation technical projects code; and 3. AWS S3 drives to store an unformatted encrypted data like documents, articles and backups.

According to embodiments herein, all Sweetch user's records are held in a secured database DB. In embodiments, all data is encrypted when stored (Data at rest). In embodiments, all data transmission is done securely using SSL. Access to the records may be based on roles and permissions, in accordance with embodiments herein.

In embodiments, sensitive data access is logged into a designated logger. According to embodiments herein, records are protected by a proper access permission to position holder. In embodiments, data is being archived regularly-different length of time for each type of records. As an archive flow, the data may be compressed, encrypted and then archived. In embodiments, Sweetch is following HIPAA requirements to retain data for 6 years. After 6 years all user data is encrypted and archived. In embodiments herein, Sweetch user data is stored on Sweetch servers only, and not shared with any external entity.

According to embodiments herein, Sweetch maintains compliance with high security standards:
Uses AWS KMS Service to encrypt all data on DB and for storing and managing the keys;
All communications are encrypted securely using SSL (https);
All personal data stored on device is done securely—the data is encrypted before stored; and
Protection against application input/output injection by using ORM technology to avoid SQL injections at any point. In addition, we constantly perform tough input validations (length, format, type etc.) to avoid any buffer overflow exploits.

According to embodiments herein, the app is based on native UI and not browser or web controls to avoid JS attacks. In embodiments, the app must first authenticate through an OAuth2 compliant authentication request. Only after a successful validation of the incoming request, the client is given a special access token it must use in every API call.

In embodiments herein, access tokens are secured against re-use, and revoked quickly. For example, the application is logged in from the first time the user logs-in and until the user logs-out, so there is no token re-use. In embodiments herein, the app disables any keystroke logging and cut-and-paste buffers for any sensitive data fields. Further, in embodiments, no private information is stored directly in the application binary. Furthermore, in an embodiment, ICSC guidance on log monitoring and management may be followed.

The terms “machine-readable medium” or “computer-readable medium” as used herein refers to any medium that participates in providing instructions to processor PRO for execution. Such a medium may take many forms, including but not limited to, non-volatile media, volatile media, and transmission media. Non-volatile media include, for example, optical or magnetic disks. Volatile media include dynamic memory. Computer-readable media can be non-transitory, for example, a floppy disk, a flexible disk, hard disk, magnetic tape, any other magnetic medium, a CD-ROM, DVD, any other optical medium, punch cards, paper tape, any other physical medium with patterns of holes, a RAM, a PROM, and EPROM, a FLASH-EPROM, any other memory chip or cartridge. Non-transitory computer readable media can have instructions recorded thereon. The instructions, when executed by a computer, can implement any of the features described herein. Transitory computer-readable media can include a carrier wave or other propagating electromagnetic signal.

Reference throughout the specification to “one embodiment”, “embodiments,” or “an embodiment” means that a particular feature, structure, or characteristic described in connection with an embodiment is included in at least one embodiment of the subject matter disclosed. Thus, the appearance of the phrases “in one embodiment” or “in an embodiment” or “in embodiments” in various places throughout the specification is not necessarily referring to the same embodiment, or different embodiments. Further, the particular features, structures or characteristics may be combined in any suitable manner in one or more embodiments. Further, it is intended that embodiments of the disclosed subject matter cover modifications and variations thereof.

Unless explicitly stated, the method embodiments described herein are not constrained to a particular order or sequence. Furthermore, all formulas described herein are intended as examples only and other or different formulas may be used. Additionally, some of the described method embodiments or elements thereof may occur or be performed at the same point in time.

While the principles of the disclosure have been made clear in the illustrative embodiments set forth above and certain features of the disclosure have been illustrated and described herein, it will be apparent to those skilled in the art that various modifications, substitutions, changes, and equivalents may be made to the structure, arrangement, proportion, elements, materials, and components used in the practice of the disclosure. It is, therefore, to be understood that the appended claims are intended to cover all such modifications and changes as fall within the true spirit of the disclosure.

Various embodiments have been presented. Each of these embodiments may of course include features from other embodiments presented, and embodiments not specifically described may include various features described herein.

It will thus be seen that the features of this disclosure have been fully and effectively accomplished. It will be realized, however, that the foregoing preferred specific embodiments have been shown and described for the purpose of illustrating the functional and structural principles of this disclosure and are subject to change without departure from such principles. Therefore, this disclosure includes all modifications encompassed within the spirit and scope of the following claims.

What is claimed is:

1. A method for prediction, prevention and management of chronic and autoimmune diseases, comprising:
receiving a plurality of inputs containing data from a user via at least one of a mobile device, an application provided thereon, or one or more connected devices;
processing the data via one or more user profiling engines and one or more user context engines,
generating, based on the one or more user profiling engines, a digital blueprint of the user;
generating, by an incremental change engine, a plurality of incremental change programs (ICPs), based at least in part on data processed from the one or more user context engines, the digital blueprint of the user, and a predefined set of goals;
assembling, by an interaction engine, based at least in part on the plurality of ICPs, the predefined set of one or more user goals, and the digital blueprint of the user, a specific interaction to be delivered to the user; and
delivering the specific interaction to the user in accordance with an optimal time and domain of intervention.

2. The method as in claim 1, wherein the digital blueprint of the user comprises a set of unique traits and features that define the user; and
wherein the set of unique traits comprises behavioral traits and physiological traits.

3. The method as in claim 1, wherein the user context engine comprises a behavioral state estimator and a situational event detector.

4. The method as in claim 1, wherein a given incremental change program facilitates a single goal or multiple goals of the predefined set of one or more user goals intra-domain, or cross-domain.

5. The method as in claim 1, wherein at least one of the incremental change engine and the interaction engine receives input from a supervision system, and
wherein the supervision system is configured to enforce one or more pre-defined policies and implements one or more pre-defined evidence-based intervention programs.

6. The method as in claim 1, wherein the specific interaction is at least one of: a specific message, a content recommendation, a trigger, a recommendation, a reminder, an alert, an averter, a reward, a challenge, a competition, or a badge.

7. The method as in claim 1, further comprising automatically detecting one or more recurring habits or patterns of future behaviors; and
providing one or more alerts recommending alternative actions in response.

8. The method as in claim 1, further comprising:
receiving historical data comprising one or more of the user's medical history and behavior over a predefined period of time, a disease current state, or a required treatment,
constructing, using one or more machine learning algorithms, a set of predictive models, based on the historical data; and
generating, by implementing an individualized quantified risk prediction engine, a unique index score for the user for a specific future timeframe, based at least in part on the set of predictive models.

9. A system for prediction, prevention and management of chronic and autoimmune diseases, comprising:
memory storing computer program instructions; and
one or more processors configured to execute the computer program instructions to:
receive a plurality of inputs containing data from a user via at least one of a mobile device, an application provided thereon, or one or more connected devices;
process the data via one or more user profiling engines and one or more user context engines;
generate, based on the one or more user profiling engines, a digital blueprint of the user;
generate, by an incremental change engine, a plurality of incremental change programs (ICPs), based at least in part on data processed from the one or more user context engines, the digital blueprint of the user, and a predefined set of one or more user goals;
assemble, by an interaction engine, based at least in part on the plurality of ICPs, the predefined set of one or more user goals, the digital blueprint of the user, a specific interaction to be delivered to the user; and
deliver the specific interaction to the user in accordance with an optimal time and domain of intervention.

10. The system as in claim 9, wherein the digital blueprint of the user comprises a set of unique traits and features that define the user; and wherein the set of unique traits comprises behavioral traits and physiological traits.

11. The system as in claim 9, wherein a given incremental change program facilitates a single goal or multiple goals of the predefined set of one or more user goals intra-domain, or cross-domain.

12. The system as in claim 9, wherein at least one of the incremental change engine and the interaction engine receives input from a supervision system, and
wherein the supervision system is configured to enforce one or more pre-defined policies and implements one or more pre-defined evidence-based intervention programs.

13. The system as in claim 9, wherein the specific interaction is at least one of: a specific message, a content recommendation, a trigger, a recommendation, a reminder, an alert, an averter, a reward, a challenge, a competition, or a badge.

14. The system as in claim 9, further configured to:
receive historical data comprising one or more of the user's medical history and behavior over a predefined period of time, a disease current state, or a required treatment,
construct, using one or more machine learning algorithms, a set of predictive models, based on the historical data; and
generate, by implementing an individualized quantified risk prediction engine, a unique index score for the user for a specific future timeframe, based at least in part on the set of predictive models.

15. A non-transitory computer readable medium having instructions recorded thereon for prediction, prevention and management of chronic and autoimmune diseases, the instructions when executed by a computer having at least one programmable processor cause operations comprising:
receiving a plurality of inputs containing data from a user via at least one of a mobile device, an application provided thereon, or one or more connected devices;
processing the data via one or more user profiling engines and one or more user context engines;
generating, based on the one or more user profiling engines, a digital blueprint of the user;
generating, by an incremental change engine, a plurality of incremental change programs (ICPs), based at least in part on data processed from the one or more user context engines, the digital blueprint of the user, and a predefined set of one or more user goals;
assembling, by an interaction engine, based at least in part on the plurality of ICPs, the predefined set of one or more user goals, the digital blueprint of the user, a specific interaction to be delivered to the user; and
delivering the specific interaction to the user in accordance with an optimal time and domain of intervention.

16. The computer readable medium as in claim 15, wherein the digital blueprint of the user comprises a set of unique traits and features that define the user; and wherein the set of unique traits comprises behavioral traits and physiological traits.

17. The computer readable medium as in claim 15, wherein a given incremental change program facilitates a single goal or multiple goals of the predefined set of one or more user goals intra-domain, or cross-domain.

18. The computer readable medium as in claim 15, wherein at least one of the incremental change engine and the interaction engine receives input from a supervision system, and
wherein the supervision system is configured to enforce one or more pre-defined policies and implements one or more pre-defined evidence-based intervention programs.

19. The computer readable medium as in claim 15, wherein the specific interaction is at least one of: a specific message, a content recommendation, a trigger, a recommendation, a reminder, an alert, an averter, a reward, a challenge, a competition, or a badge.

20. The computer readable medium as in claim 15, further comprising:
receiving historical data comprising one or more of the user's medical history and behavior over a predefined period of time, a disease current state, or a required treatment,
constructing, using one or more machine learning algorithms, a set of predictive models, based on the historical data; and
generating, by implementing an individualized quantified risk prediction engine, a unique index score for the user for a specific future timeframe, based at least in part on the set of predictive models.
